SEQUENCE LISTING (21 sequences)

SEQ ID NO: 1 - DNA, 27 bp, Artificial Sequence
  Synthetic construct
  misc_feature (27)..(27): 3′ end labelled with Atto 488
aggtcgccgcccggttgggtgggttgg

SEQ ID NO: 2 - DNA, 12 bp, Artificial Sequence
  Synthetic construct
gggcggcgacct

SEQ ID NO: 3 - DNA, 33 bp, Artificial Sequence
  Synthetic construct
  misc_feature (13)..(13): FAM
  misc_feature (33)..(33): DABCYL
aggtcgccgccctccaacggttggtgtggttgg

SEQ ID NO: 4 - DNA, 15 bp, Artificial Sequence
  Synthetic construct
ccaaccacaccaacc

SEQ ID NO: 5 - DNA, 15 bp, Artificial Sequence
  Synthetic construct
ccaaccgcaccaacc

SEQ ID NO: 6 - DNA, 15 bp, Artificial Sequence
  Synthetic construct
ccaaccgcaccgacc

SEQ ID NO: 7 - DNA, 15 bp, Artificial Sequence
  Synthetic construct
ccagccgcaccgacc

SEQ ID NO: 8 - DNA, 12 bp, Artificial Sequence
  Synthetic construct
aggtcgccgccc

SEQ ID NO: 9 - DNA, 50 bp, Artificial Sequence
  Molecular Beacon for Let 7a miRNA
  misc_feature (12)..(14): Alexa 488 Label
  misc_feature (50)..(50): Dabcyl
aggtcgccgccctctgcaacaactatacaacctactacctcagttgcaga

SEQ ID NO: 10 - DNA, 50 bp, Artificial Sequence
  Molecular Beacon for miR-21 miRNA
  misc_feature (12)..(14): Alexa 488 label
  misc_feature (50)..(50): Dabcyl label
gggcggcgaccttcttggactcaacatcagtctgataagctagtccaaga

SEQ ID NO: 11 - DNA, 50 bp, Artificial sequence
  Molecular Beacon for miR-375 miRNA
  misc_feature (12)..(14): Alexa 488 Label
  misc_feature (50)..(50): Dabcyl Label
aggtcgccgccctccgtacgtcacgcgagccgaacgaacaaacgtacgga

SEQ ID NO: 12 - DNA, 50 bp, Artificial Sequence
  Molecular Beacon for miR-141 miRNA
  misc_feature (12)..(14): Alexa 488 Label
  misc_feature (50)..(50): Dabcyl Label
gggcggcgaccttccggcacccatctttaccagacagtgttagtgccgga

SEQ ID NO: 13 - DNA, 22 bp, Artificial Sequence
  Let-7amiRNA DNA
tgaggtagtaggttgtatagtt

SEQ ID NO: 14 - DNA, 22 bp, Artificial Sequence
  miR-21 miRNA DNA
tagcttatcagactgatgttga

SEQ ID NO: 15 - RNA, 22 nt, Homo sapiens
ugagguaguagguuguauaguu

SEQ ID NO: 16 - RNA, 22 nt, Homo sapiens
uagcuuaucagacugauguuga

SEQ ID NO: 17 - RNA, 22 nt, Homo sapiens
uuuguucguucggcucgcguga

SEQ ID NO: 18 - RNA, 22 nt, Homo sapiens
uaacacugucugguaaagaugg

SEQ ID NO: 19 - RNA, 22 nt, Homo sapiens
ugagguaguagauugugugguu

SEQ ID NO: 20 - RNA, 22 nt, Homo sapiens
uaacacugucugguaacgaugu

SEQ ID NO: 21 - RNA, 22 nt, Artificial Sequence
  Scrambled sequence for miR-141
augagugagagauacgcuucua

(19) United States
(12) Patent Application Publication
(10) Pub. No.: US 2022/0372577 A1
(43) Pub. Date: Nov. 24, 2022

(54) ANALYTE DETECTION METHOD

(71) Applicant: IMPERIAL COLLEGE OF SCIENCE, TECHNOLOGY AND MEDICINE, London (GB)

(72) Inventors: Joshua Benno EDEL, London (GB); Shenglin CAI, London (GB); Jasmine Y Y SZE, London (GB); Aleksandar IVANOV, London (GB)

(21) Appl. No.: 17/434,548

(22) PCT Filed: Feb. 27, 2020
(86) PCT No.: PCT/GB2020/050479

§ 371 Date: Aug. 27, 2021

(30) Foreign Application Priority Data
Feb. 27, 2019 (GB) 1902630.1

(51) Int. Cl. C12Q 1/6886

(52) U.S. Cl. CPC C12Q 1/6886; C12Q 1/6818

(57) ABSTRACT

The invention relates to methods of detecting and/or quantifying analytes in a sample, as well as methods of detecting mutations and/or polymorphisms in nucleic acid molecules. The methods include: providing at least one carrier nucleic acid molecule comprising at least one single-stranded region; providing at least one detection element comprising: at least one fluorophore, at least one fluorescence quencher that quenches spectroscopic detection of the fluorophore; at least one analyte-binding moiety; and at least one nucleic acid moiety that binds to a single stranded region on the carrier nucleic acid molecule; wherein the detection element is configured such that in the absence of the analyte the fluorophore is quenched by the fluorescence quencher and upon analyte binding to the analyte-binding moiety fluorescence is restored; binding these with an analyte to form a complex; translocating the complex through a nanopore via voltage-driven translocation and monitoring time-dependent current response; irradiating the nanopore with radiation that excites the fluorophore and monitoring radiation emissions of the fluorophore over time; and comparing the signals from time-dependent current response and emission over time.

### FIELD OF INVENTION

The present invention provides methods for the detection of analytes, including but not limited to biological molecules such as proteins or peptides, via simultaneous electrochemical and optical measurements.

### BACKGROUND

The ability to detect and characterise molecular interactions, especially for small molecules is crucial in understanding many biological processes, allowing us to discover biomarkers for disease diagnosis and screening drug candidates. An accurate, efficient method to quantify these small molecules in solution as well as a high-throughput manner will have a significant impact in biological research and drug discovery.

Current methods for measuring these molecules rely on bulk measurement and labelled methods, including ELISA and radiolabelling, which do not provide kinetic information as the majority of them are endpoint assays. In addition, the use of these labelled molecules could affect the native properties of the analyte of interest and are prone to interference caused by nonspecific binding, leading to inaccurate conclusions.

Nanopores are a class of label-free single molecule sensors where individual molecules can be distinguished based on the ionic current. The detection principle relies on the transient modulations of the ionic current as single molecules pass through the nanoscale pore under the influence of an applied potential. Yet, the method remains challenging when detecting small biomolecules, especially for molecules that are much smaller than that of nanopore size, in addition to its heterogeneous charge and fast transport through nanopores. Although chemical modification around the pore, smaller pore size with high bandwidth amplifier and the use of carrier methods have been explored to slow down the translocation time and increase the capture rate, individual electrical detection still lacks the spatial/positional resolution regarding the molecules of interest.

In contrast, optical methods such as fluorescence correlation spectroscopy (FCS), and total internal reflection fluorescence (TIRF), have been used in characterising these molecules in solution and in real-time. However, conventional single molecule FCS techniques are usually performed on a diffusion-based, microscope glass-slide where molecules of interest diffuse in and out of the detection volume in a stochastic manner and in three-dimensions. To control the delivery of the molecule and enhance the spatial resolution of this technique, the target molecules should align well with the laser beam and pass through the detection volume within one dimension.

Recently, solid-state nanopores combined with single molecule fluorescence techniques addressed some of the above limitations and have demonstrated proof-of concept study for detection of DNA and DNA-PNA complex [1-3]. These approaches generally relied on the requirement of labelling of target molecules and would not be practical to pursue for further development for multiple small molecules/protein detection due to additional fabrication steps and imperfect labelling. In addition, these studies only provide limited insight into the feasibility of the binding, rather than the kinetics of the binding/biomolecular interaction. The ability to detect these small biomolecules binding to a specific recognition region would be particularly useful for precise target screening along a specific location within the development. Finally, the approach was limited to small, planar solid state nanopore, which sets restrictions on the size of the target interests and the pore size range of the setup.

### DESCRIPTION OF INVENTION

To address the challenges found in the prior art, the advantages of both nanopore sensing and single-molecule fluorescence spectroscopy can be combined, to enable an efficient strategy for detection using nanopores.

Accordingly, in a first aspect the present invention provides a method of detecting one or more analytes in a sample, the method comprising:
a. providing at least one carrier nucleic acid molecule comprising at least one single-stranded region;
b. providing at least one detection element comprising:
   i. at least one fluorophore;
   ii. at least one quencher that modifies spectroscopic detection of the fluorophore;
   iii. at least one analyte-binding moiety; and
   iv. at least one nucleic acid moiety that binds to a single stranded region on the carrier nucleic acid molecule;
   v. wherein the detection element is configured such that in the absence of the analyte the fluorophore is quenched by the fluorescence quencher and upon analyte binding to the analyte-binding moiety fluorescence is restored;
c. contacting the carrier nucleic acid molecule and detection element with the sample to form a carrier nucleic acid molecule/detection element/analyte complex;
d. providing a nanopore through which the carrier nucleic acid/detection element/analyte complex may be translocated;
e. translocating the carrier nucleic acid/detection element/analyte complex through the nanopore via voltage-driven translocation and monitoring time-dependent current response;
f. irradiating the nanopore with radiation that excites the fluorophore and monitoring radiation emissions of the fluorophore over time; and
g. comparing the signals from time-dependent current response and emission over time;

wherein a simultaneous signal in both time-dependent current response and emission over time indicates the binding of the analyte to the detection element.

In one embodiment there is provided a method of detecting one or more analytes in a sample, the method comprising:
a. providing at least one carrier nucleic acid molecule comprising at least one single-stranded region;
b. providing at least one detection element comprising:
   i. at least one fluorophore;
   ii. at least one fluorescence quencher that quenches spectroscopic detection of the fluorophore;
   iii. at least one analyte-binding moiety; and
   iv. at least one nucleic acid moiety that binds to a single stranded region on the at least one carrier nucleic acid molecule;
   v. wherein the at least one detection element is configured such that in the absence of the one or more analytes the at least one fluorophore is quenched by the at least one fluorescence quencher and upon analyte binding to the at least one analyte-binding moiety fluorescence is restored;
c. contacting the at least one carrier nucleic acid molecule and at least one detection element with the sample to form at least one carrier nucleic acid molecule/detection element/analyte complex;
d. providing a nanopore through which the at least one carrier nucleic acid/detection element/analyte complex may be translocated;
e. translocating the at least one carrier nucleic acid/detection element/analyte complex through the nanopore via voltage-driven translocation and monitoring time-dependent current response;
f. irradiating the nanopore with radiation that excites the at least one fluorophore and monitoring radiation emissions of the at least one fluorophore over time; and
g. comparing the signals from time-dependent current response and emission over time;

wherein a simultaneous signal in both time-dependent current response and emission over time indicates the binding of the one or more analytes to the at least one detection element.

The carrier nucleic acid and detection element are configured such that they will form a complex via nucleic acid hybridisation. This complex is then detectable via voltage-driven translocation through a nanopore. In the presence of the analyte, the detection element/analyte complex also binds to the carrier nucleic acid to form the nucleic acid/detection element/analyte complex. In the presently described method, simultaneous detection of a translocation event in the time-dependent current response and a fluorescence signal indicates the binding of the analyte to the analyte-binding moiety of the detection element.

It will be appreciated that the term “simultaneously” should be interpreted as occurring at substantially the same moment. Depending on the calibration of the experimental set up, some variation in the precise times of optical and electrochemical detection will be expected. Such variations are within the understanding of the skilled person and are encompassed within the meaning of the term “simultaneously” as used herein. As used herein, the term “synchronized” and simultaneous may be used interchangeably to refer to events as occurring at substantially the same moment.

Advantageously, the combination of electrochemical and optical detection avoids false positives sometimes observed in nanopore translocation methods. Without wishing to be bound by any particular theory, it is believed that these false positive signals are due to translocation of alternative conformations of carrier nucleic acid, for example a folded or dimerised molecule. The present method avoids these false positives by requiring a simultaneous optical detection event.

The detection element comprising a fluorophore, fluorescence quencher and analyte-binding moiety is configured such that in the absence of the analyte the fluorophore is quenched by the fluorescence quencher and upon analyte binding fluorescence is restored. Such molecules may be referred to herein as molecular beacons and their general structure will be known to those of skill in that art. Exemplary structures include, but are not limited to, nucleic acids with stem-loop structures in which a portion of the molecule binds to itself in the absence of an analyte. Upon binding to their target analyte, molecular beacons undergo a conformational change which changes the physical distance between the fluorophore and the fluorescence quencher, resulting in a measurable change in the fluorescence signal of the fluorophore. Any suitable molecular beacon may be used in the method described herein.

Thus in certain embodiments, the detection element comprises a molecular beacon (MB). In certain embodiments the detection element is an MB.

The analyte-binding moiety of the detection element may be a single stranded portion that is complementary to a nucleic acid analyte. Binding of the single stranded portion to the analyte results in the aforementioned conformation change and measurable change in the fluorescence signal.

Preferably the analyte-binding moiety of the detection element is an aptamer. Aptamers are oligonucleotide sequences (ssDNA or RNA) with the ability to non-covalently bind to targets with high specificity and affinity. Aptamer targets include, but are not limited to, a nucleic acid, protein, carbohydrate, fatty acid or another molecule of interest. Molecules of interest may also include, but are not limited to, small molecules having a molecular weight of less than about 50 kDa. The use of a detection element that can bind to the analyte in this way is advantageous because it does not require chemical modification of the analyte.

In certain embodiments, the number of detection elements corresponds to the number of the single stranded regions of the at least one carrier nucleic acid molecule. This is to say that each single stranded region of the carrier molecule is capable of binding to a single corresponding detection element.

The presently described method may also be applied to multiplex detection by the addition of multiple carrier nucleic acids and detection elements. In such a scenario, each detection element will have an analyte-binding moiety that binds to a particular analyte.

In certain embodiments the method comprises providing at least two carrier nucleic acid molecules wherein each carrier nucleic acid molecule has a different molecular weight and/or length. Without being bound by theory the size, for example molecular weight and/or chain length of carrier nucleic acids, can be differentiated by individual electrical events due to the dwell time of the carrier nucleic acid or carrier nucleic acid/detection element/analyte complex within the nanopore during translation, which may be proportional to the size of the carrier nucleic acid.

It is also possible to differentiate carrier nucleic acids and/or carrier nucleic acid/detection element/analyte complexes by analysing current observed during translation events.

During the translocation events, carrier nucleic acids, in complex or not, may be un-coiled to match the cross-section of a nanopore and then transported through the pore, displacing a portion of electrolytes, leading to a measurable change in the ionic current. Thus, the dwell time for larger carrier nucleic acids and any complexes formed therewith may be longer than that for smaller carrier nucleic acids and/or complexes formed therewith due to the larger carrier nucleic acids and/or complexes having a longer residence time in the nanopore due to carrier nucleic acid threading through the nano pore.

Furthermore, the current change observed between different sized carrier nucleic acids may be relative to the overall charges of the carrier nucleic acids in translocation, which is called an event charge deficit (ECD). If the log of the ECD is calculated and plotted it is possible to distinguish different carrier nucleic acids based on size (e.g. molecular weight or length).

This discrimination for different lengths or sizes of carrier nucleic acids has the advantage of enabling multiple carrier nucleic acids to have different single-stranded regions which are capable of each binding different corresponding detection elements, therefore allowing for multiple different analytes to be simultaneously detected and distinguished within a single sample. This allows for multiplex detection. In certain embodiments each detection element may have a different fluorophore, resulting in a different fluorescence signal for each analyte.

In addition, multiplex detection may be achieved with a single carrier nucleic acid having multiple single-stranded regions, to which multiple different detection elements may bind. Again, in this scenario it is envisioned that each detection element will have a different fluorophore such that distinct signals for each detection element (and thus each analyte) are generated

Accordingly, there is also provided a method in which:
i. the carrier nucleic acid has at least two single stranded regions; and
ii. a number of detection elements corresponding to the number of single stranded regions is provided;
wherein the analyte-binding moieties in each detection element may bind to the same or to different analytes and wherein each detection element has a different fluorophore.

Accordingly, in a further aspect of the invention there is provided a method of detecting two or more analytes in a sample, the method comprising:
a. providing at least one carrier nucleic acid molecule comprising at least two single-stranded regions;
b. providing at least two detection elements each comprising:
   i. at least one fluorophore;
   ii. at least one fluorescence quencher that quenches spectroscopic detection of the respective fluorophore;
   iii. at least one analyte-binding moiety; and
   iv. at least one nucleic acid moiety that binds to at least one of the single stranded regions on the at least one carrier nucleic acid molecule;
   v. wherein each detection element is configured such that in the absence of a respective one of the two or more analytes the at least one fluorophore is quenched by the at least one fluorescence quencher and upon analyte binding to the at least one analyte-binding moiety fluorescence is restored;
c. contacting the at least one carrier nucleic acid molecule and each detection element with the sample to form at least one carrier nucleic acid molecule/detection element/analyte complex; wherein each detection element is bound to a respective single stranded region of the carrier nucleic acid molecule and a respective analyte;
d. providing a nanopore through which the at least one carrier nucleic acid/detection element/analyte complex may be translocated;
e. translocating the at least one carrier nucleic acid/detection element/analyte complex through the nanopore via voltage-driven translocation and monitoring time-dependent current response;
f. irradiating the nanopore with radiation that excites at least one of the fluorophores and monitoring radiation emissions of at least one of the fluorophores over time; and
g. comparing the signals from time-dependent current response and emission over time;

wherein a simultaneous signal in both time-dependent current response and emission over time indicates the binding of the one or more analytes to the at least one detection element.

Preferably, in the method described above the analyte-binding moieties in each detection element bind to different analytes. Preferably, each detection element will have a different fluorophore such that distinct signals for each detection element (and thus each analyte) are generated. In certain embodiments the irradiating (step f.) may be repeated utilising radiation that excites all of the two or more fluorophores and each fluorophore emits a different fluorescence signal (for example different wavelength and/or energy) thus allowing detection of each analyte to be distinguished.

In certain embodiments the two or more fluorophores may have different excitation energies and irradiating may further comprises a second step of irradiating utilising a second radiation that excites a different fluorophore than that of the first irradiation.

One possible application of the invention according to certain aspect is the detection of biomarkers, such as for example cancer biomarkers. Early-stage screening of cancers may be challenging due to the lack of appropriate biomarkers regarding all types of cancers, and universal protein markers are often only detectable when most therapeutic interventions are less effective. Recent advances showed that microRNAs (miRNAs), a class of short (typically 18 to 23 nucleotides) non-coding endogenous RNAs, can play critical roles in various physiological and pathological processes, such as for example embryonic differentiation, cellular proliferation and apoptosis, haematopoiesis, and cardiac hypertrophy, by means of binding to the 3′ untranslated regions of target message RNAs (mRNAs) and degrading them or silencing the expression of relevant proteins.

Thus, miRNAs may be of high value as biomarkers for identifying abnormal cell proliferation and/or tissue differential state, which can be used as hallmarks of cancers, particularly in the early stages of cancer. Expression of miRNAs has been reported to be closely linked to different levels of cancer progression. Advantageously, miRNAs show higher levels of stability than other biomarkers in bodily fluids (for example, blood, urine, and saliva) and hence could serve as potential biomarkers for minimally invasive assessment of cancers prior to treatments and/or investigative techniques such as biopsies and/or imaging scans.

Accordingly, in certain embodiments, the analyte may be a cancer biomarker.

In certain embodiments, the one or more analytes comprise DNA or RNA. In certain embodiments, the one or more analytes comprises a microRNA (miRNA). In certain embodiments, the miRNA is one or more of miR-141, miR-375, Let 7a and/or miR-21. In certain embodiments, the one or more analytes are cancer biomarkers. In certain embodiments, the cancer is selected from one or more of lung, breast, ovarian, colorectal and/or prostate cancer. In certain embodiments, the sample is a bodily fluid. In certain embodiments, the sample is human serum.

Accordingly, in a further aspect the invention provides an in vitro method of diagnosing and/or assessing cancer in a patient the method comprising:
a. providing at least one carrier nucleic acid molecule comprising at least one single-stranded region;
b. providing at least one detection element comprising:
   i. at least one fluorophore;
   ii. at least one fluorescence quencher that quenches spectroscopic detection of the fluorophore;
   iii. at least one cancer biomarker-binding moiety; and
   iv. at least one nucleic acid moiety that binds to a single stranded region on the carrier nucleic acid molecule;
   v. wherein the detection element is configured such that in the absence of a cancer biomarker the fluorophore is quenched by the fluorescence quencher and upon cancer biomarker binding to the cancer biomarker-binding moiety fluorescence is restored;
c. contacting the carrier nucleic acid molecule and detection element with a sample containing the cancer biomarker to form a carrier nucleic acid molecule/detection element/cancer biomarker complex;
d. providing a nanopore through which the carrier nucleic acid/detection element/cancer biomarker complex may be translocated;
e. translocating the carrier nucleic acid/detection element/cancer biomarker complex through the nanopore via voltage-driven translocation and monitoring time-dependent current response;
f. irradiating the nanopore with radiation that excites the fluorophore and monitoring radiation emissions of the fluorophore over time; and
g. comparing the signals from time-dependent current response and emission over time;

wherein a simultaneous signal in both time-dependent current response and emission over time indicates the binding of the cancer biomarker to the detection element.

Detection of a simultaneous signal in both time-dependent current response and emission over time therefore can indicate whether a patient has cancer and/or provide an assessment of the stage of cancer. It should be understood that the term “assessing” is used herein to refer to determination of a stage of cancer. For example, determining the extent to which a cancer has developed, grown and/or spread and/or whether a patient has active cancer or the cancer is in remission.

Diagnosing and/or assessing cancer using single miRNA may be difficult since the variation of expression in different disease stages can be very small and sometimes can overlap. Furthermore, one specific miRNA can act as biomarker for multiple diseases rather than an indicator for a specific type of cancer. Current technologies are challenging for profiling multiple miRNAs using a one-sample test and also are time-consuming and can be error-prone.

Therefore aspects of the current invention can improve diagnosis and/or assessment of cancer by detecting multiple miRNAs simultaneously with a single sample.

MiR-141 and miR-375 are two typical miRNAs that have been reported to be upregulated in the tumour or circulation of prostate cancer patients. Let 7a and miR-21 RNA are commonly observed RNA sequences that are involved in a series of tumour regulations and are frequently investigated as biomarkers for many cancers, such as lung, breast, ovarian and colorectal cancer.

One further possible application of the methods described herein is the detection of mutations and/or polymorphisms in nucleic acids of interest. Polymorphisms as used herein refers to a discontinuous genetic variation resulting in the occurrence of several different forms or types of individuals among the members of a single species. The term “mutation” as used herein refers to a change that occurs in a DNA or RNA sequence of an organism, either due to mistakes during DNA replication and/or transcription or as the result of environmental factors such as UV light and cigarette smoke and/or cancer. Mutations maybe one or more deletions, replacements and/or additions of nucleic acid bases within a given sequence.

By quantifying the fraction or percentage of simultaneous events (S) over all the translocations of carriers, it is possible to determine the concentrations of individual analytes. The value of S equates to a percentage of the occurrences of simultaneous signal in both time-dependent current response and fluorescence emission over time for every translocation through the nanopore of a carrier that occurs. That is to say, S can be calculated by the number of simultaneous events divided by the total number of electrical events that are recorded by a means for monitoring the time-dependent current response from the nanopore.

In certain embodiments, the analyte-binding moiety comprises a nucleic acid, the sample is a control sample and the one or more analytes comprise a control nucleic acid comprising a sequence complimentary to the nucleic acid sequence of the at least one analyte-binding moiety; and the method further comprises:
repeating steps a. to g. of the first aspect with a second sample wherein the one or more analytes comprise a target nucleic acid;
calculating a percentage of the occurrences of the simultaneous signal in both time-dependent current response and emission over time over all electrical signals obtained for the at least one carrier nucleic acid/detection element/control nucleic acid complex (S);
calculating a percentage of the occurrences of the simultaneous signal in both time-dependent current response and emission over time over all electrical signals obtained for the at least one carrier nucleic acid molecule/detection element/target nucleic acid complex (S′);

wherein a value of S′ lower than the value of S indicates the presence of one or more mutations and/or nucleotide polymorphisms.

Accordingly, one aspect of the present invention provides a method of detecting one or more mutations and/or polymorphisms in a sample, the method comprising:
a. providing at least one carrier nucleic acid molecule comprising at least one single-stranded region;
b. providing at least one detection element comprising:
   i. at least one fluorophore;
   ii. at least one quencher that modifies spectroscopic detection of the fluorophore;
   iii. at least one binding moiety comprising a nucleic acid; and
   iv. at least one nucleic acid moiety that binds to a single stranded region on the carrier nucleic acid molecule;
   v. wherein the detection element is configured such that in the absence of a control nucleic acid or target nucleic acid the fluorophore is quenched by the fluorescence quencher and upon control nucleic acid or target nucleic acid binding to the binding moiety fluorescence is restored;
c. contacting the carrier nucleic acid molecule and detection element with a control sample, the control sample comprising the control nucleic acid and wherein the control nucleic acid comprises a nucleic acid sequence complimentary to the nucleic acid sequence of the at least one binding moiety to form a carrier nucleic acid molecule/detection element/control nucleic acid complex;
d. providing a nanopore through which the carrier nucleic acid/detection element/control nucleic acid complex may be translocated;
e. translocating the carrier nucleic acid/detection element/control nucleic acid complex through the nanopore via voltage-driven translocation and monitoring time-dependent current response;
f. irradiating the nanopore with radiation that excites the fluorophore and monitoring radiation emissions of the fluorophore over time; and
g. comparing the signals from time-dependent current response and emission over time;

wherein a simultaneous signal in both time-dependent current response and emission over time indicates the binding of the analyte to the detection element;
h. calculating a percentage of the occurrences of the simultaneous signal in both time-dependent current response and emission over time over all electrical signals obtained for the at least one carrier nucleic acid/detection element/control nucleic acid complex (S);
i. providing the at least one carrier nucleic acid molecule comprising at least one single-stranded region and the at least one detection element as defined in a. and b.;
j. contacting the carrier nucleic acid molecule and detection element with a test sample, the test sample comprising the target nucleic acid, to form a carrier nucleic acid molecule/detection element/target nucleic acid complex;
k. providing a nanopore through which the carrier nucleic acid/detection element/target nucleic acid complex may be translocated;
l. translocating the carrier nucleic acid/detection element/target nucleic acid complex through the nanopore via voltage-driven translocation and monitoring time-dependent current response;
m. irradiating the nanopore with radiation that excites the fluorophore and monitoring radiation emissions of the fluorophore over time; and
n. comparing the signals from time-dependent current response and emission over time;

wherein a simultaneous signal in both time-dependent current response and emission over time indicates the binding of the target nucleic acid to the detection element;
o. calculating a percentage of the occurrences of the simultaneous signal in both time-dependent current response and emission over time over all electrical signals obtained for the at least one carrier nucleic acid/detection element/target nucleic acid complex (S′);

wherein a value of S′ lower than the value of S indicates the presence of one or more mutations and/or nucleotide polymorphisms in the target nucleic acid.

As the analyte-binding moiety comprises a nucleic acid having a sequence complementary to the sequence of the control nucleic acid, the control nucleic acid is able to bind to the nucleic acid of the binding moiety via nucleic acid base pairing.

The simultaneous signal in both time-dependent current response and emission over time for the control sample can therefore provide a control or baseline measurement.

Upon providing the test sample, if the target nucleic acid comprises any mis-matched base pairs in comparison to the nucleic acid sequence of the nucleic acid of the binding moiety there will be a reduced level of base pairing between the target nucleic acid and the nucleic acid of the binding moiety. This reduction of base pairing leads to a reduced number of detection elements that will be in a conformation that allows for the fluorophore to fluoresce. Therefore there will be reduced occurrences of the simultaneous signal in both time-dependent current response and emission over time. This reduction in the simultaneous signal therefore indicates the presence of one or more mis-matched bases between the target nucleic acid and the nucleic acid of the binding moiety.

In certain embodiments, the target nucleic acid may not bind to the nucleic acid of the binding moiety and therefore no simultaneous signal is produced.

In certain embodiments, the target nucleic acid and the nucleic acid of the binding moiety have at least 1, at least 2, at least 3 or more mismatched nucleic acids.

In certain embodiments, the target nucleic acid and the nucleic acid of the binding moiety have at least 99% sequence homology, at least 95% sequence homology, or at least 90% sequence homology.

In certain embodiments, the target nucleic acid comprises RNA and/or DNA.

In certain embodiments, the nucleic acid of the binding moiety comprises RNA and/or DNA.

One of the advantages of simultaneous (e.g. synchronised) detection is that the concentration of analytes can be quantified by quantifying the percentage (S) of simultaneous optical events over all electrical signals. As used herein, the term “all electrical signals” refers to the total number of electrical events that are recorded by a means for monitoring the time-dependent current response from the nanopore over the time period of measurements taken.

According to one aspect of the invention there is provided a method of quantifying a concentration of an analyte in a sample, the method comprising:
a. providing at least one carrier nucleic acid molecule comprising at least one single-stranded region;
b. providing at least one detection element comprising:
   i. at least one fluorophore;
   ii. at least one fluorescence quencher that quenches spectroscopic detection of the fluorophore;
   iii. at least one analyte-binding moiety; and
   iv. at least one nucleic acid moiety that binds to a single stranded region on the carrier nucleic acid molecule;
   v. wherein the detection element is configured such that in the absence of the analyte the fluorophore is quenched by the fluorescence quencher and upon analyte binding to the analyte-binding moiety fluorescence is restored;
c. contacting the carrier nucleic acid molecule and detection element with a sample comprising an analyte to form a carrier nucleic acid molecule/detection element/analyte complex;
d. providing a nanopore through which the carrier nucleic acid/detection element/analyte complex may be translocated;
e. translocating the carrier nucleic acid/detection element/analyte complex through the nanopore via voltage-driven translocation and monitoring time-dependent current response;
f. irradiating the nanopore with radiation that excites the fluorophore and monitoring radiation emissions of the fluorophore over time; wherein a simultaneous signal in both time-dependent current response and emission over time indicates the binding;
g. comparing the signals from time-dependent current response and emission over time;

wherein a simultaneous signal in both time-dependent current response and emission over time indicates the binding of the analyte to the detection element;

h. calculating a percentage of the occurrences of the simultaneous signal in both time-dependent current response and emission over time over all electrical signals (S);

i. comparing S to one or more reference values of S to determine the concentration of analyte.

In certain embodiments, the one or more reference values of S are obtained by:
j. carrying out steps a. to h. wherein the sample is a control sample comprising a known concentration of the analyte;
repeating step j. at least two times, wherein the known concentration of analyte is increased or decreased.

The value of S can be calculated for a number of known concentrations of analyte in a control sample. By increasing or decreasing the concentration of analyte in a control sample and calculating subsequent values of S for each concentration of analyte it is possible to produce a plot of concentration of analyte against S. This plot can then be used as a standard measure of S for a given concentration of analyte.

In certain embodiments the standard plot may be plotted using one or more logarithmic scales. Using logarithmic scales may provide a plot having a straight line relationship between S and concentration of analyte.

Once a plot has been produced the value of S for a test sample can be calculated and this value can be compared to the standard plot. By marking the intercept of the value of S obtained for the test sample with the line defined on the standard plot, the corresponding intercept with the concentration of analyte can be read from the plot. Thereby providing a concentration of analyte in the test sample.

Accordingly, the method of quantifying a concentration of an analyte in a test sample comprises:
a. providing at least one carrier nucleic acid molecule comprising at least one single-stranded region;
b. providing at least one detection element comprising:
   i. at least one fluorophore;
   ii. at least one fluorescence quencher that quenches spectroscopic detection of the fluorophore;
   iii. at least one analyte-binding moiety; and
   iv. at least one nucleic acid moiety that binds to a single stranded region on the carrier nucleic acid molecule;
   v. wherein the detection element is configured such that in the absence of the analyte the fluorophore is quenched by the fluorescence quencher and upon analyte binding to the analyte-binding moiety fluorescence is restored;
c. contacting the carrier nucleic acid molecule and detection element with a control sample comprising a known concentration of the analyte to form a carrier nucleic acid molecule/detection element/analyte complex;
d. providing a nanopore through which the carrier nucleic acid/detection element/analyte complex may be translocated;
e. translocating the carrier nucleic acid/detection element/analyte complex through the nanopore via voltage-driven translocation and monitoring time-dependent current response;
f. irradiating the nanopore with radiation that excites the fluorophore and monitoring radiation emissions of the fluorophore over time; wherein a simultaneous signal in both time-dependent current response and emission over time indicates the binding of the analyte to the detection element;
g. calculating a percentage of the occurrences of the simultaneous signal in both time-dependent current response and emission over time over all electrical signals (S);
h. repeating steps a. to g. at least two times, wherein the known concentration of analyte in the control sample is increased or decreased in order to produce a calibration standard graph of concentration of analyte versus S;
i. providing the at least one carrier nucleic acid molecule comprising at least one single-stranded region and the at least one detection element as defined in a. and b; and
j. contacting the carrier nucleic acid molecule and detection element with a test sample comprising the analyte to form a carrier nucleic acid molecule/detection element/analyte complex;
k. providing a nanopore through which the carrier nucleic acid/detection element/analyte complex may be translocated;
l. translocating the carrier nucleic acid/detection element/analyte complex through the nanopore via voltage-driven translocation and monitoring time-dependent current response;
m. irradiating the nanopore with radiation that excites the fluorophore and monitoring radiation emissions of the fluorophore over time; wherein a simultaneous signal in both time-dependent current response and emission over time indicates the binding of the analyte to the detection element;
n. calculating a percentage of the occurrences of the simultaneous signal in both time-dependent current response and emission over time over all electrical signals (S);
o. determining concentration of analyte in the test sample by comparing the value of S calculated in step n. to the calibration standard graph.

The nanopore may be any suitable nanopore through a nucleic acid can be translocated while monitoring time-dependent current response. Preferably the nanopore is at the tip of a nanopipette. Nanopipettes may be manufactured by any suitable method available to the trained person. Quartz nanopipettes are particularly preferred as they are relatively easy to fabricate and do not introduce extra electrical noise or optical background.

Voltage-driven translocation through the nanopore may be achieved via any suitable means.

Irradiation of the nanopore and monitoring of radiation emissions may be carried out by any suitable means, preferably confocal microscopy. Where the nanopore is at the tip of a nanopipette, irradiation may be achieved from any incident angle.

In addition, the present inventors have unexpectedly found that the combination of electrochemical detection using nanopores and optical detection improves the performance of optical detection. Again, without wishing to be bound by any particular theory, it is believed that the constrained physical volume of the nanopore reduces diffusion of the fluorophore in and out of the optical detection volume. Accordingly, the dwell time of the molecule is increased, leading to a corresponding increase in signal.

Also described is a carrier nucleic acid molecule comprising a fluorophore, fluorescence quencher and a sequence which binds to an analyte, configured such that upon analyte binding the carrier nucleic acid undergoes a conformation change that results in a measurable change in the fluorescence signal of the fluorophore.

In a third aspect there is provided an apparatus for detection of an analyte characterised in that it is adapted to use the method of the first aspect. Preferably the apparatus will comprise:
at least one volume for receiving a sample;
at least one nanopore, adapted to be in contact with the at least one volume for receiving a sample;
at least one source of potential difference, adapted to apply a potential difference across the at least one nanopore;
means for monitoring the time-dependent current response from the nanopore;
at least one source of electromagnetic radiation adapted to illuminate the at least one nanopore;
at least one detection means adapted to detect fluorescence radiation signals arising from the at least one nanopore; and
means for the comparison of signals from the means for monitoring the time-dependent current response from the nanopore and the signals form the at least one detection means, adapted to identify simultaneous events.

Preferred embodiments described in respect of the first aspect are also contemplated in respect of the above-described further aspects.

### DESCRIPTION OF DRAWINGS

FIG. 1. Translocation of custom MB and binding to the unlabelled target molecules using synchronised opto-electron detection. A. MB translocated through the nanopipette and only electrical signal was observed. B and C. With addition of complementary oligonucleotide and proteins, it binds to the complementary/specific sequence within the custom MB and resulted in the opening of the hairpin where fluorescence is restored with the quencher, as observed by the average number of fluorescent molecules (no. of photos) in the detection volume, as shown in the optical signals.

FIG. 2. Experimental set up for synchronized opto-electro detection.

FIG. 3. Schematic of the experimental setup. A quartz nanopipette was mounted on a coverslip and aligned to the objective of a custom-built confocal fluorescence microscope and adapted to incorporate a custom Faraday cage and headstage connected to an A&M 2400 Amplifier. Optical measurements were obtained using 488 nm laser excitation which was beam expanded (BE) to ensure backfilling of the objective. The laser was reflected by a dichroic mirror (DM) and introduced into the back aperture of a 60× water immersion objective (Obj.). The fluorescence from the tip of the nanopipette was collected by the same objective and passed through the same DM followed by alignment to a confocal pinhole (PH). Another DM was used to split the light into two channels (green: 500-580 nm; red: 640-800 nm) and focus the light using a lens (L) onto two avalanche photodiode detectors (APDs). The electrical and optical data were collected via two DAQ cards and triggered to record simultaneously using a custom written Labview program.

FIG. 4. SEM images of a nanopipette. (a) SEM image showing the geometry and the taper of a quartz nanopipette. (b) SEM image of the tip with a nanopore diameter of 21±2 nm

FIG. 5. Current-voltage curves for the nanopipettes. I-V curves obtained using 100 mM KCl Tris-EDTA buffer (pH=8.0). The conductance of the nanopipettes was estimated to be 3.7±0.2 nS (n=20).

FIG. 6. Effect of laser power on the electrical noise. Power spectrum density (PSD) plots obtained for the nanopipettes prior to and after illumination with a 488-nm laser (Power=198±6 μW). Data was obtained using an applied bias of −500 mV and a solution containing 100 mM KCl TE buffer (pH=8.0).

FIG. 7: Single Molecule electro-optical detection of 5 kbp DNA labelled with YOYO-1. a Photon and current time traces for the translocation of 100 μM 5 kbp DNA-YOYO-1 in 100 mM KCl, 10 mM Iris-HCl, 1 mM EDTA buffer (pH=8). The resample time for the photon time trace is 500 μs and the filter frequency for the current time trace is 10 kHz. b Percent synchronisation, c signal to noise and d dwell time as a function of applied voltage for the electrical (blue circles) and optical (brown squares) channels respectively. Error bars represent the accumulation of statistics from at least 3 different nanopipettes.

FIG. 8: Electro-optical time traces for the translocation of 5 kbp DNA-YOYO-1 at low voltages. At lower voltages (−80, −60 and −40 mV), events were only detected in the optical channel. The sample used was 5 kbp DNA-YOYO-1 (100 pM) in 100 mM KCl TE buffer (pH=8.0). Scale bars (optical, top): vertical 200 photons, horizontal 0.5 s. (electrical, bottom): vertical 20 pA, horizontal 1 s. The resampling time for the photon time trace was 500 μs and the electrical time trace was filtered at 10 kHz. Laser power is 90±3 μW.

FIG. 9: Comparison of signal and noise distributions for the optical data shown in FIG. 2c. Peak amplitudes of 470±122, 418±130, 451±154 and 454±151 counts/0.5 ms were obtained at voltages of −300, −200, −150 and −100 mV, respectively. The background signal remained very low and relatively constant (4.8, 4.7, 4.7, and 4.7 counts/0.5 ms respectively). The sample used was 5 kbp DNA-YOYO-1 (100 pM) in 100 mM KCl TE buffer (pH=8.0).

FIG. 10: Comparison of dwell time distributions for 5 kbp DNA-YOYO-1. Mean dwell time for the electrical data (a-d) are 0.65±0.29, 0.86±0.21, 1.01±0.28, and 1.37±0.38 ms and optical (e-h) data are 28.5±10.4, 40.6±14.8, 44.8±15.1, and 50.3±14.9 ms, for voltages of −300, −200, −150 and −100 mV. All data were recorded in 100 mM KCl TE buffer (pH=8.0).

FIG. 11: Schematic for hybridisation of λ-DNA and its complementary oligo. A 27 mer oligo (5′-AGGTCGCCGCCC GGTTGGGTGGGTTGG-Atto 488-3′) (SEQ ID NO: 1) was used with the 3′ end modified to incorporate an Atto 488 label. The underlined sequence, was used to bind to the sticky end of the 5′ end of λ-DNA (5′-GGGCGGCGACCT-3′) (SEQ ID NO: 2).

FIG. 12: Single-molecule and single fluorophore sensitivity. a Photon and current time traces for the translocation of λ-DNA bound to a fluorescently labelled DNA oligo in 100 mM KCl, buffer (pH=8). The oligo is labelled with Atto 488 at its 3′ end, and the sequence is 5′-AGGTCGCCGCCC GGTTGGGTGGGTTGG-Atto 488-3′, (SEQ ID NO: 1) which contains 12 complementary bases (underlined) to enable binding to λ-DNA. Synchronised events are highlighted with a dashed box. b Scatter plots for dwell time versus current amplitude/intensity for both electrical and c optical measurements. Data for both synchronised and non-synchronized events are shown. d A binding assay was demonstrated using a DNA carrier modified with a biotinylated oligo which can then bind to streptavidin. Translocation experiments were performed at −300 mV bias in 100 mM KCl buffer (pH=8). A final DNA carrier concentration of 10 pM was used and incubated with Dylight 488-conjugated streptavidin at varying concentrations at room temperature. e Binding assay for a 10 pM DNA carrier concentration incubated with increasing streptavidin concentration ranging from 0 to 100 pM. Error bars indicate the standard deviation for data obtained from 3 different nanopipettes.

FIG. 13: Comparison of typical electro-optical events for the translocations of (a) λ-DNA, (b) dye-oligo, and (c) λ-DNA-oligo-dye complex under a potential bias of −300 mV. The optical dwell time and total photons (peak area) detected significantly increased when the small oligos binding to the λ-DNA carrier, whereas the electrical signals (dwell time and peak amplitude) remain similar before and after the binding occurred. Laser power: 198±6 μW

FIG. 14: Control experiments for the translocations of λ-DNA and oligos only. Synchronised electrooptical detection was carried out using λ-DNA (10 pM) (a) and oligos (100 pM) (b). As expected, only independent electrical or optical signals can be observed without synchronisation. Both experiments were carried out at 100 mM KCl, TE buffer (pH=8.0) at an applied voltage of −300 mV. The resampling time for the photon time trace was 1 ms and the electrical time trace was filtered at 10 kHz. Laser power: 198±6 μW.

FIG. 15: Dwell time and current amplitude distributions for the translocation of λ-DNA (control). The mean dwell time (a), and peak amplitude (b) were 5.0±2.2 ms and 61.5±21.3 pA, respectively. Data was recorded using 10 pM λ-DNA in 100 mM KCl, TE buffer (pH=8.0) and applying a potential of −300 mV.

FIG. 16: Voltage-dependence on dwell time and peak height/intensity for the translocation of the λ-DNA-oligo complex. The electrical dwell times and peak amplitudes remain constant for synchronised and non-synchronised events, (a) and (b). Optical dwell times were approximately 5-fold slower for the synchronised events, and the total photon counts were 10-fold higher, (c) and (d). All data were recorded using 100 mM KCl, TE buffer (pH=8.0). The shaded area is equivalent to 1 standard deviation.

FIG. 17: Synchronised electro-optical detection of streptavidin using biotinylated carriers. (a) A 20-second electro-optical time trace for the translocation of a biotinylated carrier (10 pM) bound to streptavidin (20 pM, Dylight 488 conjugated) at a bias of −300 mV (100 mM KCl TE buffer, pH=8.0). Laser power 198±6 μW (b) Three typical electro-optical signals were observed corresponding to the translocations of (i) biotinylated carrier, (ii) streptavidin, and (iii) carrier-streptavidin complex, respectively. Scale bars (optical): vertical 25 counts/ms, horizontal 10 ms. (electrical): vertical 20 pA, horizontal 10 ms. The resampling time for the photon time trace was 1 ms and the electrical time trace was filtered at 10 kHz.

FIG. 18. Comparison of dwell time and peak amplitude distributions for the translocation of biotinylated DNA carriers bound to streptavidin from −150 to −300 mV. All data were recorded in 100 mM KCl TE buffer (pH=8.0).

FIG. 19: Control experiments for the translocations of λ-DNA and biotinylated carrier. Synchronised electro-optical detection was carried out for (a) λ-DNA (10 pM) (b) biotinylated carriers (10 pM). As expected, the only signal at the electrical detection channel could be observed. Both of the data were recorded in 100 mM KCl TE buffer (pH=8.0). The resampling time for the photon time trace was 1 ms and the electrical time trace was filtered at 10 kHz. Laser power 198±6 μW.

FIG. 20: Label-free detection of DNA oligos and proteins using molecular beacons. Photon and current time traces are shown for the translocation of a DNA MB-Carrier, b DNA MB-Carrier-cDNA, and c DNA MB-Carrier-Thrombin. The DNA carrier, cDNA, and thrombin concentrations were 10 pM, 50 pM, 30 nM respectively. Translocations were recorded at −300 mV bias in a buffer of 100 mM KCl (pH=8). d It was possible to determine the orientation of the complex translocating through the nanopore by characterising the e fractional position for the onset of the optical signal relative to the onset of the electrical signal. f Percent synchronisation between the optical and electrical channels for cDNA bound to the MB-carrier along with controls including single, double and triple base mismatches. g A Binding affinity of 3.7 pM was calculated by fitting a Hill binding model for cDNA as a function of % synchronization. h Percent synchronisation between the optical and electrical channels for thrombin bound to the MB-carrier along with controls. A Binding affinity of 5.0 pM was calculated by fitting a Hill binding model for thrombin as a function of % synchronization which was in agreement with existing bulk methods. Error bars in f and h were determined using data obtained from a minimum of 3 different nanopipettes.

FIG. 21: Schematic for the preparation of MB-Carrier and its binding to the nucleic acid/protein. (a) The molecular beacon was incorporated into a DNA carrier through hybridization to the 3′ end of λ-DNA (5′-GGGCGGCGACCT-3′)(SEQ ID NO: 2). The sequence of MB is as follows: 5′-AGGTCGCCGCCC-T(FAM)-CCAAC GGTTGGTGTGGTTGG-DABCYL-3′ (SEQ ID NO: 3). The underlined bases are complementary to the 3′ end of λ-DNA. The bases in italics and bold represent the stem of the MB. The aptamer sequence incorporated into the MB targeting thrombin is shown in red. (b) When the MB-Carrier binding to a cDNA (15 bases, 5′-CCA ACC ACA CCA ACC3′) (SEQ ID NO: 4), the stem-loop was opened, the distance between the fluorophore probe and the quencher increases leading to restored fluorescence, while binding to single-base mismatch (SM), double-bases mismatch (DM), or triple-bases mismatch (TM) DNA, no fluorescence was observed. Sequences used are SM (5′-CCAACC GCA CCA ACC-3′)(SEQ ID NO: 5), DM (5′-CCA ACC GCA CCG ACC-3′)(SEQ ID NO: 6), TM (5′-CCA GCC GCA CCG ACC-3′)(SEQ ID NO: 7). The mismatched bases are bolded and underlined. (c) The addition of thrombin has led to the opening up of the loop to form a G-quadruplex structure and extend the distance between fluorophore and quencher, resulting in the emission of fluorescence. However, in a control protein mixture of lysozyme, trypsin, α-synuclein and insulin, no binding to the MB-carrier did not occur, and no fluorescence could be observed.

FIG. 22: Dwell time vs Intensity scatter plot for synchronised and non-synchronised events for the MBCarrier with cDNA/thrombin. (a) When incubating the MB-carrier (10 pM) with the cDNA or thrombin, similar electrical dwell time and current amplitude were observed for non-synchronised (5.4±2.5 ms, 60.4±28.3 pA, N=162) and synchronised events (5.6±2.6 ms, 65.4±34.2 pA, N=147). (b) However, optical detection shows a clear increase in both dwell time and photon counts when comparing synchronized (N=147) and non-synchronized events (N=87). (c) With thrombin, similar dwell times and amplitudes were observed for non-synchronised (4.3±2.0 ms, 56.2±26.6 pA, N=196) and synchronised events (4.9±2.3 ms, 66.0±34.8 pA, N=91. (d) Much like for cDNA, optical detection shows a clear increase in both dwell time and photon counts when comparing synchronized (N=91) and non-synchronized events (N=77). All data was recorded at −300 mV bias in 100 mM KCl TE buffer (pH=8.0).

FIG. 23: Control experiments for the MB-carrier incubating with mismatched DNA. Photon and current time traces for the translocation of 10 pM MB-Carrier incubating with (a) single-mismatch (SM), (b) doublemismatch (DM), and (c) triple-mismatch (TM) DNA, 50 pM for each. The concentration of MB-carrier was 10 pM. In these particular examples, no synchronised events were observed over the trace duration (20 s), which indicated the high selectively of the MB-carrier. All translocations were recorded at −300 mV bias using 100 mM KCl TE buffer (pH=8). The resampling time for the photon time trace was 1 ms and the electrical time trace was filtered at 10 kHz. Laser power 198±6 μW.

FIG. 24: Control experiments for the MB-carrier incubating with protein mixture. Photon and current time traces for the translocation of 10 pM MB-Carrier incubating with a protein mixture (lysozyme, α-synuclein, trypsin and insulin, 10 μM for each). Same here, no synchronised events were observed indicated the selectively of the MB-carrier incorporated with TBA. Data were recorded at −300 mV bias using 100 mM KCl TE buffer (pH=8.0). Laser power 198±6 μW.

FIG. 25: Sensing of cDNA and protein targets in human serum and urine. a Photon time traces for the detection of cDNA bound to the MB-Carrier in (i) 0.1 M KCl (pH=8), (ii) 0.1M KCl+5% human serum, (iii) and 0.1M KCl+10% urine. Conventional confocal single molecule methods were used (i.e. droplet on a coverslip). b Comparable traces to those shown in a using a nanopore. A significant decrease in background fluorescence is observed in part due to the solution being confined to inside the nanopipette. The reservoir outside the nanopipette only contains a 0.1 M KCl buffer solution. Translocation experiments were performed at −300 mV and in all cases, the laser power was 193±6 μW. c Photon and current time traces are shown for the translocation of (i) thrombin in 5% serum, (ii) MB-Carrier in 5% serum, and (iii) MB-Carrier bound to thrombin in 5% serum. The MB-Carrier and thrombin concentration was 30 pM and 1 nM respectively. (iv) Percent synchronisation between the optical and electrical channels for thrombin bound to the MB-carrier at concentrations ranging from 0.1-100 nM. Error bars indicate the standard deviation for data obtained from 3 different nanopipettes.

FIG. 26: Thrombin concentration dependence in human serum. Intensity time traces were recorded at −300 mV bias using 100 mM KCl TE buffer (pH=8.0) in 5% human serum. Laser power 193±6 μW. The MB-Carrier concentration was 30 pM, and thrombin was varied between 0.1-100 nM.

FIG. 27: cDNA concentration dependence in urine. Intensity time traces were recorded at −300 mV bias using 100 mM KCl TE buffer (pH=8.0) in 10% urine. Laser power 193±6 μW. The MB-Carrier concentration was 10 pM, and cDNA was varied between 1-100 pM.

FIG. 28: Schematic for four MB probes for Let 7a, miR-21, miR-375 and miR141.

FIG. 29: λ-DNA digestion and preparation of MB-Carriers with 10 kbp and 38.5 kbp.

FIG. 30: DNA carriers encoded with different lengths and their translocation characteristics. (a) Current-time trace for the translocation of 10 pM DNA in 100 mM KCl buffer (5 mM MgCl₂, 10 mM Tris-EDTA, pH=8.0) using a typical nanopipette (^{˜}20 nm). (b) Two typical events recorded by electrical signals, one with short dwell time and peak area is translocation for 10 kbp carriers, the other with large dwell and area is for 38.5 kbp. The distributions of dwell time, peak area, and current amplitude are shown in (c-e). The log-scale of dwell time and peak area and their plot are shown in (f-g). (h) Scatter plots of dwell time vs. the peak area shows two clear distributions for 10 kbp and 38.5 kbp, respectively.

FIG. 31: Single-molecule opto-electronic detection of miR-375 and miR-141 in the buffer. Left panel in (a-d) shows the intensities-time traces for the translocation of (a) MB-Carriers on its own, (b) MB-Carriers with miR-375 (10 pM), (c) MB-Carriers with 10 of miR-141 (10 pM), and (d) MB-Carriers with both miR-375 (10 pM) and miR-141 (10 pM). The MB-Carriers are 10 pM of MB-Carrier_{10kbp_miR-375} and MB-Carrier_{38.5kbp_miR-141}. The middle panel shows the zoom-in views of some typical events that represent the small and large carriers, respectively. The right panel shows the corresponding percentage of synchronised events over the electrical events for small and large carrier's translocations, respectively. All the translocations were performed at −300 mV in 100 mM KCl buffer (5 mM MgCl₂, 10 mM Tris-EDTA, pH=8.0). Laser power is 90±4 μW. All error bars represent the standard deviation for data measured from at least 3 different nanopipettes (n=3).

FIG. 32: Single molecule opto-electronic detection of Let 7a and miR-21 and the DNA analogues in the buffer. (a-c) Percent synchronisation for the translocation of (a) Carrier_{10kbp_miR-Let 7a} and MB-Carrier_{38.5kbp_miR-21} on their own (b) Carrier_{10kbp_miR-Let 7a} and MB-Carrier_{38.5kbp_miR-21} with the complementary DNA oligos, and (c) Carrier_{10kbp_miR-Let 7a} and MB-Carrier_{38.5kbp_miR-21} with Let 7a and miR-21 miRNAs. All the carrier and targets concentrations are 10 pM. All the translocations were performed at −300 mV in 100 mM KCl buffer (5 mM MgCl₂, 10 mM Tris-EDTA, pH=8.0). Laser power is 90±4 μW. All error bars represent the standard deviation for data measured from at least 3 different nanopipettes (n=3).

FIG. 33: Concentration dependence. (a-d) Photon and current-time traces for the Carrier_{10kbp_miR-375} and Carrier_{38.5kbp_miR-141} translocations with the increasing of additional miR-375 and miR-141 (from 1 pM to 100 pM). (e-f) Plots of the fraction of synchronised events as a function of miR-375/miR-141 concentrations. All the concentrations for MB-Carriers are 10 pM. The insert show log-scale of synchronised fraction versus miRNA concentrations at the range from 0.2 pM to 10 pM. These curves reveal the linear relationship between the synchronised fraction and miRNA concentrations. All the translocations were performed at −300 mV in 100 mM KCl buffer (5 mM MgCl₂, 10 mM Tris-EDTA, pH=8.0). Laser power is 90±4 μW. All error bars represent the standard deviation for data measured from at least 3 different nanopipettes (n=3).

FIG. 34: Detection at lower target concentrations using salt gradient. (a-b) The concentration dependence of synchronised fraction at the range of 0.02 pM to 1 pM using asymmetric KCl for the cis (40 mM) and trans (400 mM). Laser power is 90±4 μW. All error bars represent the standard deviation for data measured from at least 3 different nanopipettes (n=3).

FIG. 35: Specificity for single-nucleotide polymorphism (SNP) discrimination. Photon and current time traces for the translocation of Carrier_{10kbp_miR-375} and Carrier_{38.5kbp_miR-441} (10 pM) at the presence of perfectly matched sequences (Let 7a and miR-141, 10 pM) (a) and single-base mismatched (Let 7f, 10 pM) to Let 7a and double-base mismatched (miR-200a, 10 pM) to miR-141. (b). (c-d) The two population of MB-Carriers were shown clearly in the scatter plots of dwell time and peak area. (e) Percent synchronisation between optical and electrical channels for perfectly matched sequences (Let 7a and miR-141) as well as mismatched controls (Let 7f, miR-200a, and scrambled miR-141). Student's t-test; Let 7a/Let 7f (p=0.00019), miR-141/miR-200a (p=0.00044). All the translocations were performed at −300 mV in 100 mM KCl buffer (5 mM MgCl₂, 10 mM Tris-EDTA, pH=8.0). Laser power is 90±4 μW. All error bars represent the standard deviation for data measured from at least 3 different nanopipettes (n=3).

FIG. 36: Simultaneous profiling of the expressions of miR-141 and miR-375 in the circuiting serum of prostate cancer patients. (a-b) Photon and current-time traces for the translocation of Carrier_{10kbp_miR-375} and Carrier_{38.5kbp_miR-141}, in the serum from either PCa patients in remission (a) or active cancer (b). The concentration for both carriers is 10 pM and the percentage of serum was 5%. (c-d) Average percent synchronisation of miR-141 and miR-375 for 5 patients in remission and 5 patients in active cancer. (e-f) Box chart of percent synchronisation for the miR-141 and miR-375 from patients in remission or active cancer. All the boxes indicate the intervals between the 25^{th} and 75^{th} percentiles. Black lines inside the boxes indicate the medians and the squares denote the mean values. Student's t-test; miR-141 (p<0.00001), miR-375 (p<0.00001). All the translocations were performed at −300 mV in 100 mM KCl buffer (5 mM MgCl₂, 10 mM Tris-EDTA, pH=8.0) that spiked with patient serum at a ratio of 10 to 1. Laser power is 90±4 μW. All error bars represent the standard deviation for data measured from at least 3 different nanopipettes (n=3).

### DETAILED DESCRIPTION

The advantages of both nanopore sensing and single-molecule fluorescence spectroscopy can be combined, to enable an efficient strategy for small molecule detection using nanopores. For example, fluorescent probes can be used to target molecules that are difficult to detect using conventional nanopore sensing, while the combined electrical and optical signals can be used to quantify binding affinities, as well as to selectively confirm the presence of a particular biomarker. Furthermore, the analyte is spatially confined within the nanopore ensuring that the fluorescent probe is uniformly illuminated across the probe volume. This is a significant advantage compared to single molecule fluorescence correlation spectroscopy whereby the molecule diffuses in and out of the detection volume.

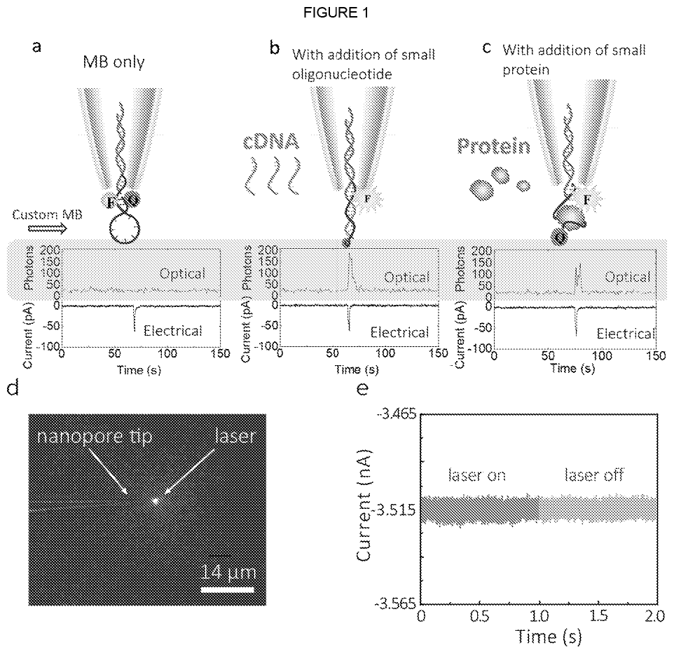

A number of groups, including our own, have already demonstrated fluorescence detection coupled to nanopores [1,4-10]. However, these studies only provide limited insight into the feasibility of molecular binding, rather than using the method to explore binding interactions. Moreover, these approaches generally rely on the labelling of the target molecule, limiting the applicability. We explore the possibility of using simultaneous detection without the need for labelling the target analyte and validate the feasibility of this strategy by detecting the presence and binding of small DNA oligomers and streptavidin to their carriers with single-fluorophore sensitivity. The fraction of synchronised events can be used to quantify the target presence and concentration. Furthermore, molecular beacons (MBs) can be designed and incorporated into the DNA carrier to screen for small proteins and complementary DNA sequences in a label-free manner, FIG. 1. MBs are short oligonucleotide fluorophore/quencher probes with “stem-loop” structures, whose sequences can be designed as needed for a range of nucleic acid binding targets [11,12]. The MBs were designed with aptamer sequences such that the corresponding protein will unravel the MB, FIG. 1, so that no labelling of the target molecule is required. The MB remains in its quenched state until the target analyte binds after which the fluorescence will then be restored.

We designed a molecular beacon (MB) incorporated DNA carrier to specifically identify short nucleic acids and small proteins using a synchronized opto-electronic platform with nanopipette. The designed MB probe have a reporter and quencher internally. When the interests of target molecule bind to the MB probe, this will open the hairpin structures, enabling the separation of the fluorophore and quencher and cause fluorescence, as observed by the emitted photos in FIGS. 1B and C. The invention enables small target detection without the need of labelling and additional preparation steps.

We used a low noise quartz nanopipettes in the proof-of-concept study. The nanopore acts as a physical gate and plays two roles: (i) to deliver molecules into the optical detection volume by modulating the applied potentials. Due to the small size of the nanopore, the molecules diffuse through to the tip and translocate to the detection volume in a one-dimensional and controlled manner, rather than in a diffusion-limited manner as in standard FCS technique. The translocation of the molecules were then monitored by recording the pulse of ionic current through nanopores as “gating” signals. The optical detection then serves as a “reporting” signal to report the fluorescence of translocated molecules.

Method: Incorporation of Nanopipette with Fluorescence Confocal Microscopy

Nanopipettes

There are a variety of solid state nanopores that could be used here to construct a synchronized opto-electronic platform. Here, a quartz nanopipette is preferred because of several advantages over the planar solid-state nanopores including: (i) ease of fabrication and (ii) no extra electrical noise or optical background are introduced, enabling high signal to noise ratio (S/R).

Single Molecule Confocal Microscopy Set Up

The optical measurements were performed using a custom-built fluorescence confocal microscope. An objective was used to introduce the laser to illuminate the exit of nanopipette tip and collect the generated fluorescence. The fluorescence emission could be detected using either electron multiplying charge coupled device (emCCD) camera or avalanche photodiodes (APD). The fluorescence could be split into two channels (500-580 nm and 640-800 nm) using a dichroic mirror (630DCXR) before detecting with APDs.

Synchronized Opto-Electronic Detection:

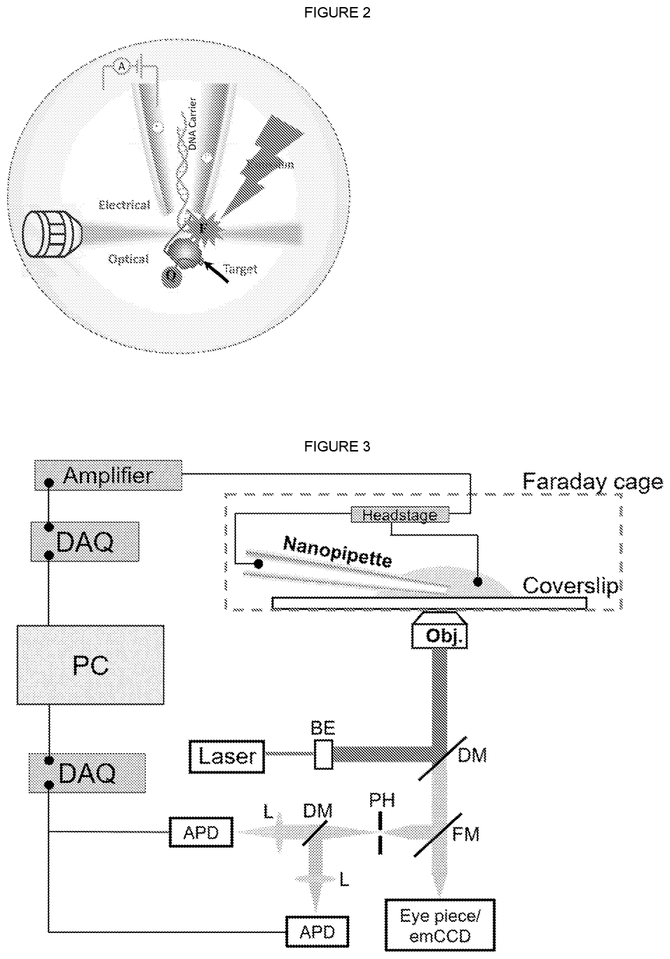

Alignment of the nanopipette to the optical detection volume is required in order to maximise the capture efficiency. Prior to each measurement, alignment of the exit of nanopipette tip with the confocal detection volume was carefully performed with the aid of emCCD camera. First, nanopipette was placed on the cover slip and its tip were fixed with tape to avoid drift. It was then set up onto the microscope perpendicular to the laser beam, followed by adjusting the objective and moving the ProScanner III stage until a clear spot/tip can be seen from the eye piece. Then, increase the laser power until a bright spot of laser were observed through a live video captured by the emCCD camera, which indicated the exact position of the confocal detection volume. Subsequently, the ProScanner III controller was utilised at the highest resolution (minimum ^{˜}10 nm per step) to scan the x-y dimension until the tip end was best aligned to the laser spot. This process was monitored in real time by the live video with emCCD camera. Finally, the z-direction was slowly adjusted with the controller (at a resolution of ^{˜}10 nm) till the very sharp tip was observed, which indicated that the nanopipette was well aligned with the laser spot. See FIG. 2 for the experimental set up.

Custom Molecular Beacon (MB) Carrier Probes

The chosen DNA carriers is a long double-stranded DNA (dsDNA). λ-DNA was selected as the base for fabricating custom MB carriers due to several characteristics such as the large molecules (48.5 kbp), leading to prolonged dwell/integration when passing through the pore/detection volume for readout; and the 12 bases overhangs which can used to hybridise different sequences and create regions for specific targets.

In this work, molecular beacons (MBs) were incorporated with λ-DNA, acting as MB carriers to identify the unlabelled targets. MBs are short oligonucleotides with stem-loop “hairpin” structures, which sequences could then be designed as needed to recognise any specific nucleic acids via simple hybridisation chemistry. Instead of direct labelling on the targets, the internally quenched fluorophores were incorporated into the MB sequences, in which fluorescence will then be restored when binding to specific targets.

This MB oligonucleotide was designed as follows: oligonucleotides that complementary to the target sequence is firstly extended by a few bases (typically 5 to 9 bases) at the 5′ end, complementing to its 3′ end to form a stem-loop structure, and further extended by 12 bases that complementary to the one of the sticky overhangs of λ-DNA. The MB-embedded oligonucleotide could be incorporated into the λ-DNA through hybridisation reaction to achieve the MB modified carrier probes.

Aptamer-Embedded MB Carrier Probes

The system could then be further extended to bind to other targets (for example proteins) by adapting corresponding aptamers into the MBs before attaching to the carrier. Aptamers are oligonucleotide sequences (ssDNA or RNA) with the ability to non-covalently bind to their targets with high specificity and affinity (Kd ranges from nM to pM). Since aptamers are obtained from a systematic evolution of ligands by exponential enrichment (SELEX) process, they could be made to be available for almost any given target molecules. Aptamers show several advantages over antibodies, for example, small size, low immunogenicity, low toxicity, ease of production and ease of modification.

This aptamer-embedded MB oligonucleotide was designed as follows: an aptamer was firstly extended by a few (5 to 9) bases at the 5′ end, complementing to its 3′ end to form a stem-loop structure, and further extended by 12 bases then complementary to the sticky overhang of λ-DNA. The aptamer-embedded MB oligonucleotide was further hybridised with λ-DNA as aforementioned method to obtain the MB-carrier.

Synchronized Detection and Quantification of Short Nucleic Acids and Proteins

This synchronized opto-electronic platform and the designed MB-incorporated carriers could be used to rapid visualization of short nucleic acids or protein with high sensitivity and selectivity. The detailed steps are as follows:

(1) Designed carriers were firstly incubated with its targets at a certain ratio in the solution of electrolyte used for translocation experiments (typically salt solution such as KCl, NaCl and LiCl).

(2) This incubation products were then introduced into the nanopipette and was set up onto the single molecule fluorescence confocal microscope. Two Ag/AgCl electrodes were placed inside (cis chamber) and outside (trans chamber) the nanopipette.

(3) After aligning as mentioned above, translocation experiments were performed by applying a potential bias between the nanopipette using certain amplifiers and corresponding current traces were recorded.

(4) The time-dependent optical signals (photon traces) for translocating molecules were detected by the fluorescence confocal microscope through APDs.

(5) When a MB-incorporated carrier without binding with its targets translocating through the nanopipette, only an individual current spike could be detected, without the following of a synchronized optical signal (photon burst) because the hairpin structure of the MB is closed.

(6) When a MB-incorporated carrier binding with its targets (could be nucleic acids or proteins) translocate through the nanopipette, a synchronous current spike accompanied with a photon burst could be detected. The stem-loop structure is in open state and allows the fluorescence emission being detected.

(7) Quantification of the synchronized events from both electrical and optical were then analysed separately using a in house Matlab code.

The present invention will be further understood by reference to the following examples

### EXAMPLES

### Example 1: Simultaneous Detection Using Nanopore and Fluorescence for Labelled Carriers; Protein Binding Detection; and Sensing of cDNA and Protein Targets in Human Serum and Urine

Materials & Methods

Chemicals and Materials

Both 5 kbp double-stranded DNA (dsDNA) and λ-DNA (48.5 kbp) with a stock concentration of 500 μg ml⁻¹ were obtained commercially from New England Biolabs. All the other DNA oligonucleotides or molecular beacon probes were synthesised by Integrated DNA Technology. Streptavidin conjugated with Dylight™ 488 was purchased from Thermo Scientific with a stock concentration of 1 mg ml⁻¹. α-thrombin was purchased from Cambridge Biosciences, UK. The fluorescent dye, YOYO-1 (1 mM in DMSO), was obtained from life technology. The stock 5 kbp dsDNA (^{˜}154 nM) was mixed with YOYO-1 at a ratio of 7.5 base pairs to 1 dye and incubated for ^{˜}30 min prior to use.

Preparation of DNA Carriers

DNA carriers used in this work were designed by hybridising of λ-DNA with either biotinylated DNA probe or a molecular beacon (MB). Briefly, DNA oligonucleotides were firstly diluted from a stock concentration (^{˜}100 μM) using a binding buffer (140 mM NaCl, 20 mM MgCl2, 10 mM Tris-EDTA buffer, pH=8.0) to 1.58 μM. 25 μl of this resulted oligonucleotide solution were then mixed with 25 μl stock λ-DNA solution and 50 μl binding buffer to achieve a total volume of 100 μl and a ratio of 1:100 of (λ-DNA: oligonucleotides). The hybridisation was then conducted by heating to 95° C. for 5 min, cooling down to 75° C. for 10 min and annealing to 25° C. at a rate of 1° C./min for 90 mins in total. The DNA carriers were then purified by removing the excess of oligonucleotide probes with the use of a 100 kDa MWCO Amicon Ultra Filter (Millipore, UK). This procedure included 6 cycles of centrifuging for 6 min at 14000 g with TE buffer (10 mM Tris-EDTA buffer, pH=8) and recovery by centrifuging at 1000 g for 2 min with turning the filter upside down. The concentration of obtained DNA carriers was determined by measuring the UV-Vis absorbance at 260 nm with a Nanodrop device (Thermo Scientific).

The MB-carrier embedded with thrombin-binding aptamer (TBA) was designed as follows 1) TBA-embedded MB oligonucleotide was designed by extending extra 5 bases on the TBA (15 mer) at the 5′ end, (this complement to its 3′ end to form a stem-loop structure) and this further extended by 12 bases (AGGTCGCCGCCC (SEQ ID NO: 8)—that is complementary to the sticky overhang of λ-DNA), to form TBA-embedded MB carrier. The TBA-embedded MB oligonucleotide was further hybridised with λ-DNA at a ratio of 100:1 and purified as the protocol above to obtain the MB-carrier. 10 pM of MB-carrier concentration was used in most of the experiments.

Fabrication of Nanopipette

Glass nanopipettes were fabricated from quartz capillaries (World Precision Instruments) as protocol reported by our group previously [13-15]. In brief, capillaries (internal diameter: 0.5 mm, external diameter: 1.0 mm, length: 7.5 cm) were plasma cleaned for ^{˜}10 min to remove any contaminated residues and pulled using a P-2000 laser-based pipette puller (Sutter Instrument, USA) to achieve two nanopipettes under the set of a two-line pulling protocol: (1) HEAT: 775; FIL: 4; VEL: 30; DEL: 170; PUL: 80, (2) HEAT: 825; FIL: 3; VEL: 20; DEL: 145; PUL: 180. This protocol generates an estimated pores with a diameter of (21 nm±2) nm. This protocol might be varied slightly from different pullers due to local temperature and humidity and should be optimised accordingly.

Optical Setup and Nanopipette Alignment

A custom-built confocal microscope was used for all optical measurement. Briefly, a 60× water immersion objective (1.20 NA, UPLSAPO 60XW, UIS2, Olympus) was used to introduce 488 nm continuous-wave solid-state laser (Sapphire 488LP, Coherent) to illuminate the exit of nanopipette tip and collect the generated fluorescence. The fluorescence irradiation was split into two channels (500-580 nm and 640-800 nm) using a dichroic mirror (630DCXR) and detected by two avalanche photodiodes (APD) (SPCM-AQR-14, PerkinElmer) respectively. Schematic representation and detailed description of the whole set up are given in FIG. 3.

Prior to each measurement, alignment of the nanopipette tip with the confocal detection volume was carefully performed with the aid of an emCCD camera (Andor). First, nanopipettes were placed on the coverslip (24×50 mm) at an angle less than 10′, and its tip was fixed with tape to avoid drift. It was then set up onto the microscope perpendicular to the laser beam, followed by raising the objective and moving the ProScanner III stage until a clear tip can be seen from the eyepiece. Then, increase the laser power until a bright laser spot was observed through a live video captured by the emCCD camera, which indicated the exact position of the confocal detection volume. Subsequently, the ProScanner III controller was utilised at the highest resolution (minimum ^{˜}10 nm per step) to scan the x-y dimension until the tip end was best aligned to the laser spot. This process was monitored in real time by the live video with emCCD camera. Finally, the z-direction was slowly adjusted with the controller at a resolution of ^{˜}50 nm till the very sharp tip was observed, which indicated that the nanopipette was well aligned with the laser spot (as shown in FIG. 1). Otherwise, we can observe a blunt of the tip with furcation when the tip is misaligned.

Translocation Experiments and Synchronised Detection

Synchronised opto-electronic detection of translocation experiments were performed from the inside to the outside of the nanopipette unless reported otherwise, where analytes together with a patch/bath electrode were introduced inside the nanopipettes (cis chamber), and a reference electrode and blank buffer were placed outside pipette tip (trans chamber). Buffer used in this work consisted of 100 mM KCl, 10 mM Tris-EDTA and 5 mM MgCl2 (pH=8). For the binding assay, DNA carriers were incubated with its targets (protein/oligos) at different ratios with a final carrier concentration of 10 pM. After introducing the solution, one Ag/AgCl electrode was inserted into the nanopipette, and the other was fixed near the pipette tip, followed by carefully placing a drop of electrolyte (^{˜}60 μl) around the nanopipette tip. After doing the alignment as mentioned above, translocation experiments were performed by applying a potential bias between the nanopipette using an A-M 2400 patch-clamp amplifier and corresponding current traces were recorded. Meanwhile, the synchronised optical signals for translocating molecules were detected by the fluorescence confocal microscope.

Data Acquisition and Analysis

A DAQ card (NI 6602, National Instruments) was coupled with the APDs for obtaining the optical data while another NI-USB 6259 DAQ card was used for the electrical data collection. The synchronisation of electrical and optical detection was triggered through a connection between these two cards and controlled by a LabView program. The electrical signal was sampled at 70 kHz and filtered at 5 or 10 kHz using a low-pass Bessel filter. The optical photon counts were collected using APD detectors with a time resolution of 10 μs.

Results

Simultaneous electro-optical measurements require a very precise alignment between the nanopore and the diffraction limited optical detection volume (^{˜}250 nm), as shown in FIG. 1. For this, quartz nanopipettes were used, a subclass of nanopores that are an ideal platform for simultaneous detection in large part due to ease of operation and due to very little or no detectable autofluorescence, unlike more common materials such as SiNₓ. Alignment was achieved by mounting the nanopipette on a coverslip inserted into a custom sample holder on a high-resolution motorised stage (with a 10 nm step size) and using an electron multiplying charge coupled device (emCCD) camera (FIG. 1) to visually align the x-y directions. The z height was fine-tuned by scanning this axis in 10 nm step sizes until scattering of the coverslip was observed using an avalanche photodiode (APD).

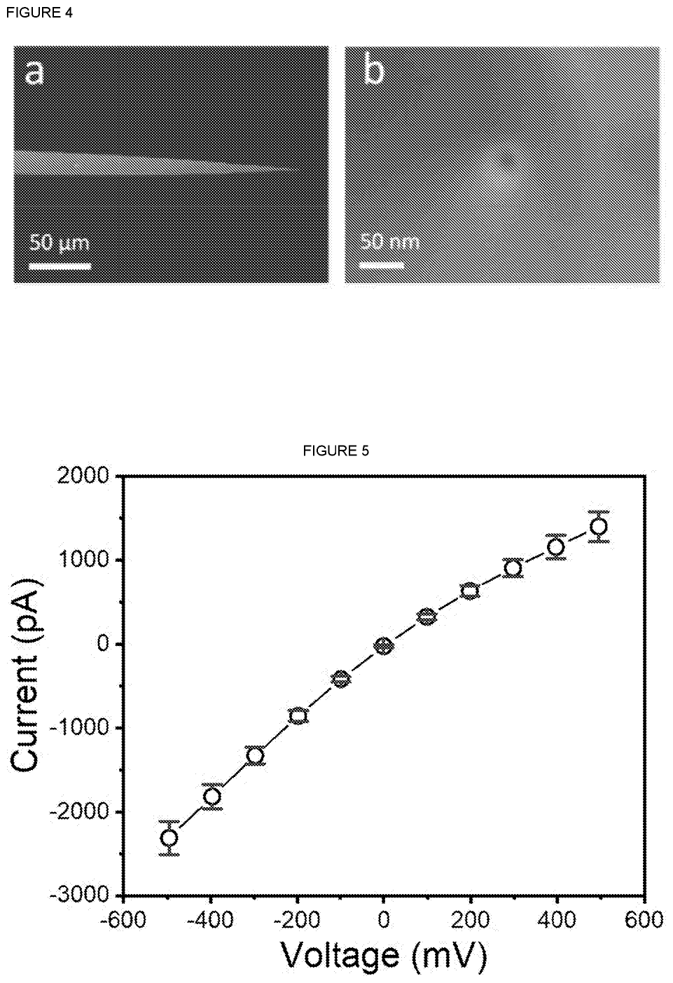

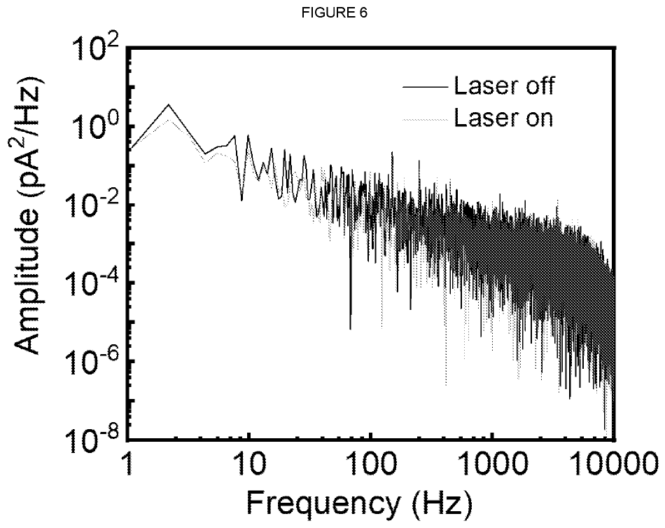

The nanopipettes were fabricated using protocols previously reported [13,14,16] yielding an average pore size of 21±2 nm(n=20), as measured by scanning electron microscopy (SEM, FIG. 4) and a nanopore conductance of 3.7±0.2 nS using 0.1 M KCl, FIG. 5. Previously it has been shown that laser illumination severely affects electrical noise characteristics due to photo-induced heating of the electrolyte and changes in surface charge on the pore surface [1,10]. In our system, we observed almost no additional electrical noise under ^{˜}198±6 μW, 488-nm laser exposure, as there was no observable increase in the baseline ionic current (FIG. 1), as well as no significant change in the power spectral densities, when the laser illumination was switched on/off, FIG. 6.

Validation of Synchronised Detection

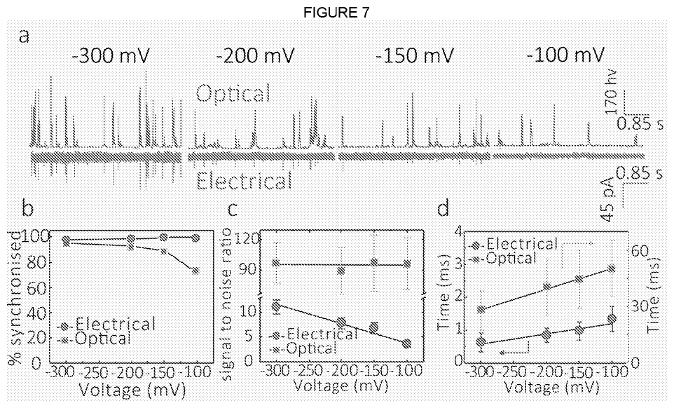

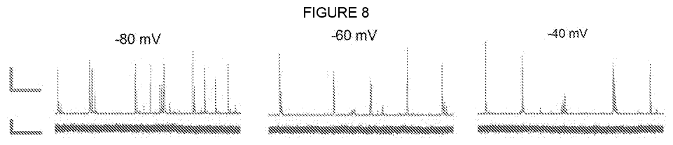

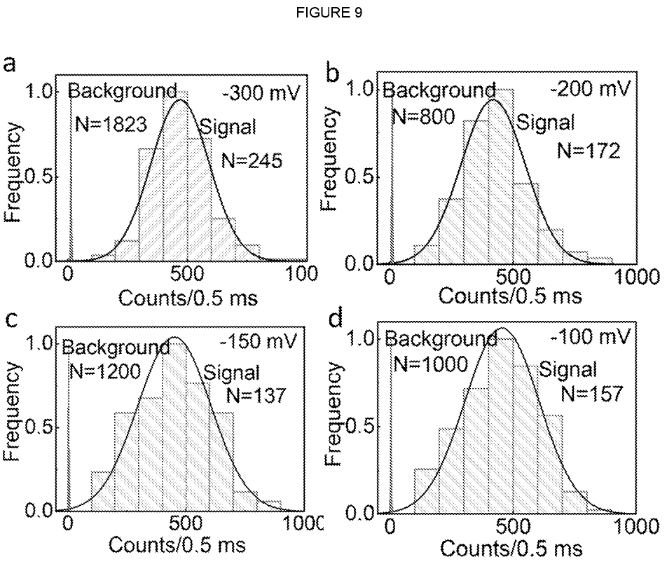

To confirm appropriate alignment, 5 Kbp DNA was fluorescently labelled with YOYO-1 and translocated through the pipette using voltages ranging from −300-−100 mV (FIG. 7) and −80-−40 mV (FIG. 8). The percentage of electrically synchronized events was high, 98.4% (n=249), 98.9% (n=174), 100% (n=144) and 100% (n=158) for −300, −200, −150 and −100 mV respectively, FIG. 7. Notably, the values are much higher than we previously reported (^{˜}92.7%) [1]. On the other hand, the synchronized percentages of total optical events are 95.3% (n=257), 93.5% (n=184) and 90.0% (n=160) for −300, −200 and −150 mV respectively, FIG. 7. It should be noted that a small underlying fraction of events appeared solely in the optical channel with much lower average intensities, FIG. 8. These events are likely due to molecules freely diffusing around or near the optical probe volume without being translocated through the nanopore. The level of synchronisation further decreases at lower voltages (71.8%, n=220 at −100 mV) in large part due to the lower peak amplitude of the electrical events causing them to be embedded within the noise. For example, the signal-to-noise (S/N) ratio decreases from 11±1.4 to 3.6±0.5 for −300 mV to −100 mV, FIG. 7. Importantly, the optical peak amplitude is not dependent on voltage and hence remains constant, 94.5±3.9 across all voltages, FIG. 7 and FIG. 9. Consequently, signals were only observed in the optical channel at lower voltages (−80 to −40 mV), FIG. 8.

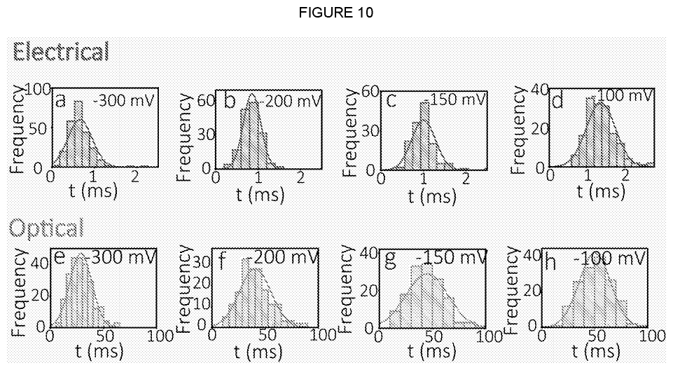

Due to confinement in the nanopore, a 40× increase in the signal to noise ratio was observed when compared to conventional diffusion-based FCS approaches. This was attributed to two factors: firstly, confinement inside the nanopipette significantly suppresses the background levels from neighbouring molecules, 204±78 photons compared to 4.9±2.2 photons respectively, FIG. 9. Secondly, the tip of the nanopipette is tightly focused, and the centre of the diffraction limited laser beam resulting in the molecule being uniformly illuminated during the translocation process. In conventional FCS, the molecule is not confined and able to diffuse freely across the Gaussian beam profile in three dimensions. Perhaps another interesting observation is in the comparison between translocation times recorded in both channels, FIG. 7 and FIG. 10. As expected, the times decrease as a function of increasing voltage; however, optical events are typically over 1 order of magnitude slower. As an intercalating dye is used, the DNA is fully labelled therefore upon exiting the nanopore the molecule recoils and freely diffuses in and around the tip. Hence the optical signal is a convolution of both the translocation process and free diffusion of the molecule. As will be described below this effect is minimised when using DNA modified with a single fluorophore in part due to the localisation of the tag.

Single Fluorophore Sensitivity

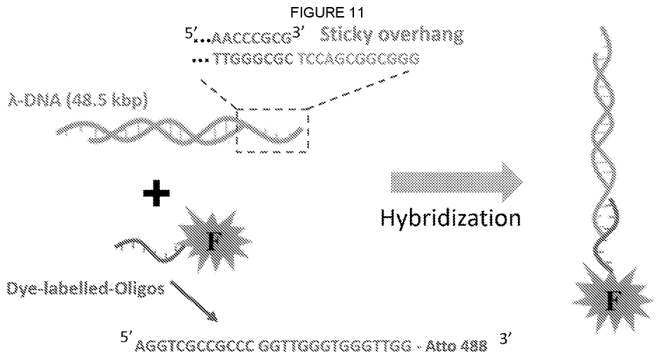

To truly take advantage of using a co-incident electro-optical detection, the sensitivity was quantified at the single fluorophore limit. A λ-DNA carrier with a 12 base overhang was used to hybridise a complementary strand (labelled with a single atto 488 dye) on the 3′ end, FIG. 11. The overhang enables facile hybridisation with any probe that can be used to selectively target and bind to an analyte [16]. In this context, a simultaneous detection strategy is useful in the sense that the nanopore effectively acts as a physical gate to deliver and detect the carriers, whereas the optical signal can be used to report on binding with a target biomolecule including ones that are much smaller than the pore dimensions.

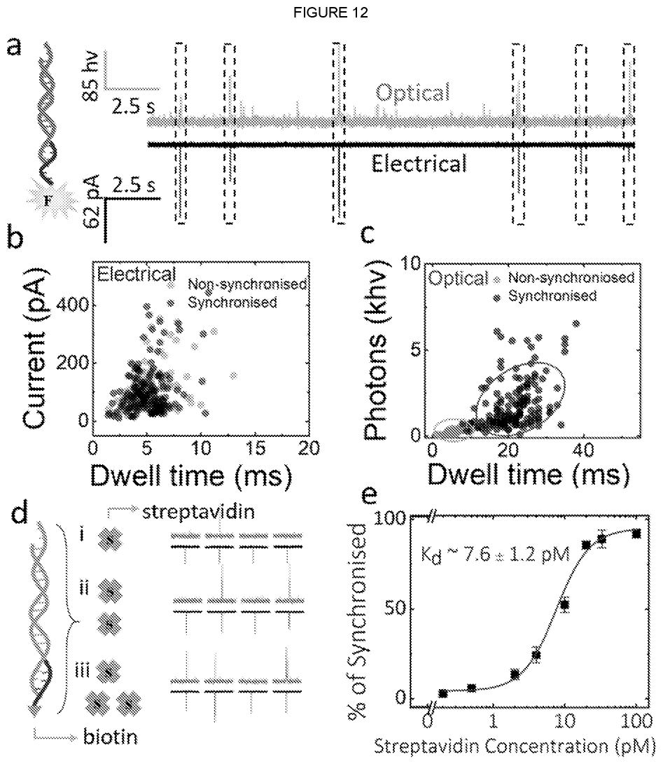

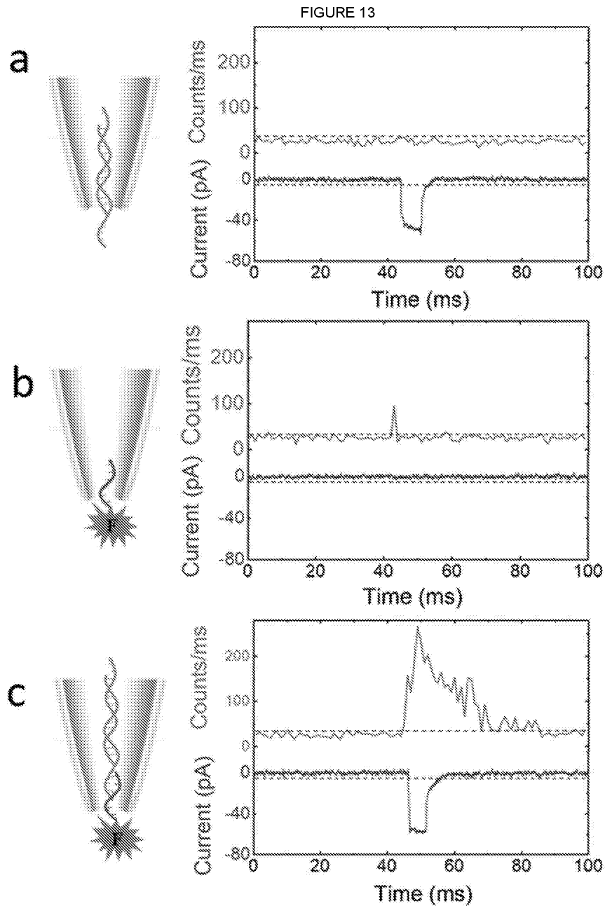

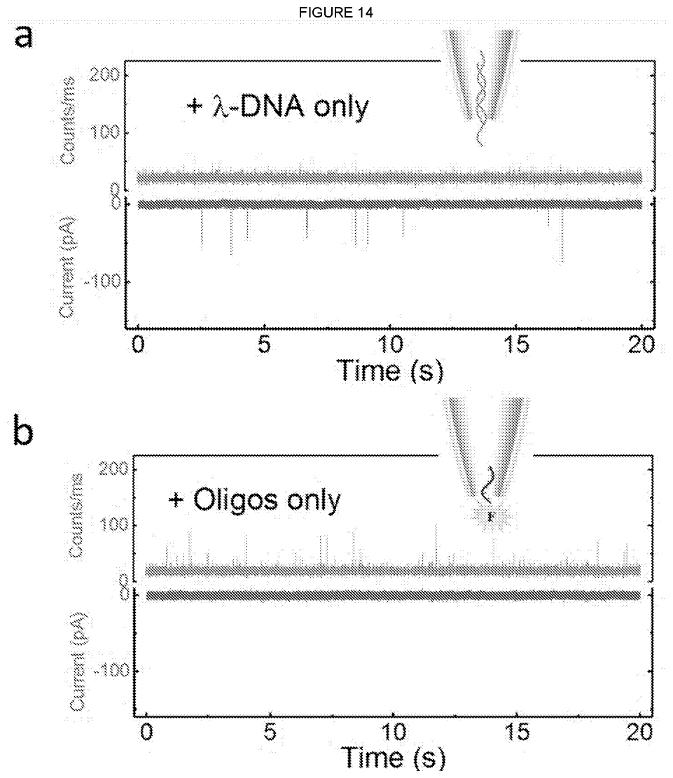

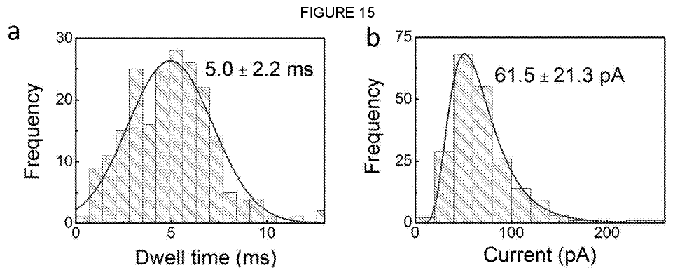

A typical intensity-time trace for a 10 pM solution of λ-DNA-oligo-dye complex obtained at a voltage of −300 mV is shown in FIG. 12. Synchronised events are highlighted with a dashed box, see also FIG. 13. Controls for both λ-DNA and the dye-oligo are shown in FIG. 14. The majority of events were coincident with a total of 287 electrical events being detected and 267 of them being synchronised with optical channel resulting in an efficiency of ^{˜}93%. We attribute the remaining 7% to be caused most likely by unsuccessful hybridisation of the oligo. Comparison of the electrical dwell times and peak amplitudes were comparable between synchronised and non-synchronised events. For example, at a voltage of −300 mV, synchronised events yields means of 5.1±1.6 ms, 63±25 pA while non-synchronized events yielded means of 5.0±1.7 ms, 65±33 pA respectively, FIG. 12. This is consistent with controls for the standard translocation of λ-DNA, FIG. 15. However, as can be seen in FIG. 12, the optical signal produced events which were at least 5× longer when comparing synchronised and non-synchronized events (21.3±4.6 vs 4.4±2.1 ms). This prolonged dwell time further confirms that the synchronised photon bursts originate from the labelled oligo binding to λ-DNA.

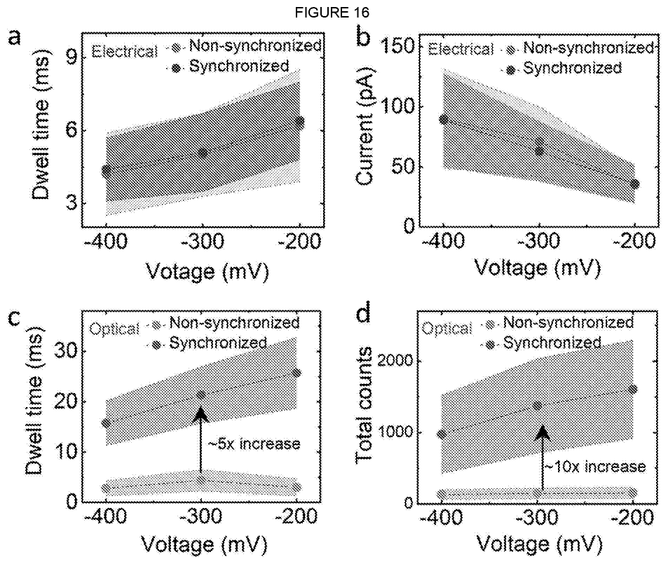

The prolonged time is in part due to the oligo-carrier complex spending more time within the optical detection volume due to the carrier slowing down the transport. This is highly advantageous as freely diffusing single molecules are often photon count limited whereas in this case a factor of 10 improvement can be made (1373±659 photons vs 145±75 photons) enabling improved statistics, FIG. 12 and FIG. 13. The increased intensity is consistent with smaller molecules diffusing away more quickly as well as due to the larger molecule spending more time in a tightly focused detection volume. A more detailed analysis of voltage dependence on dwell time and peak amplitude/intensity is shown in FIG. 16 where similar improvements are seen at both higher and lower voltages.

An Electro-Optical Single-Molecule Protein Binding Assay

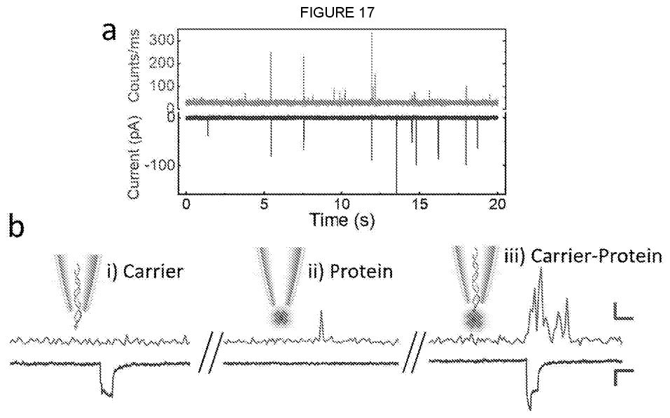

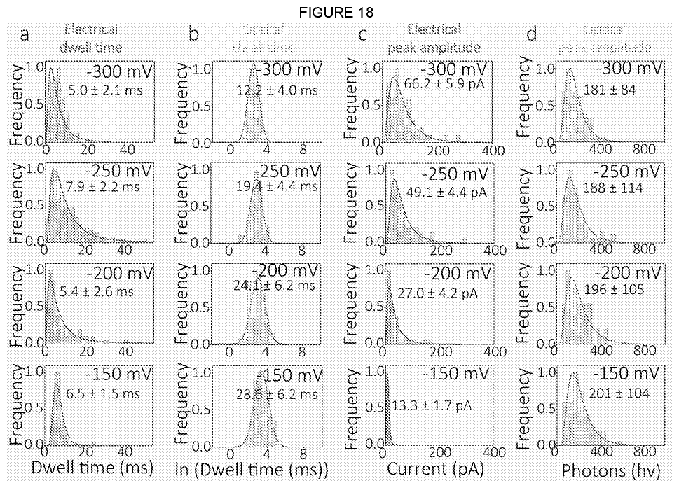

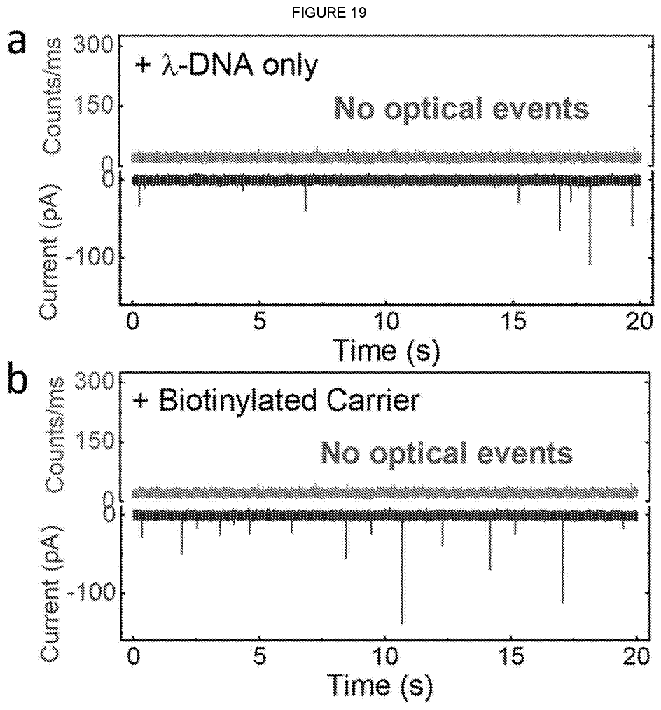

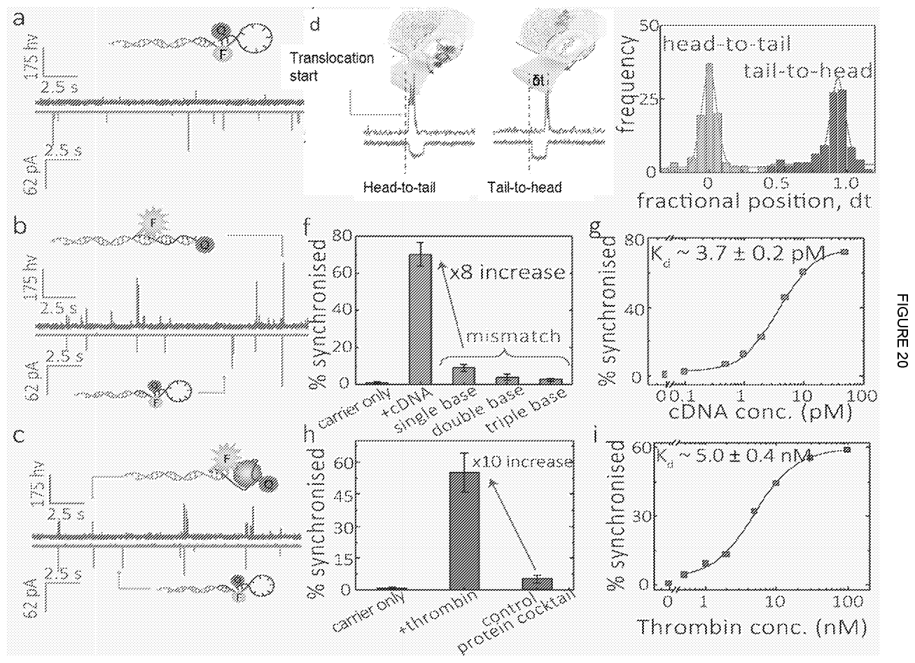

The platform can be further extended to perform an electro-optical binding assay. A 12-base biotinylated oligonucleotide (complementary to the 3′ end of λ-DNA) was hybridised to the λ-DNA (see Methods for details) to serve as the carrier for detection of the target protein, streptavidin. The biotinylated carriers were incubated with fluorescently labelled streptavidin (Dylight 488) at a ratio of 1:2 followed by translocation at a final concentration of 10 pM. As expected, the free carriers produced a signal in the electrical channel, streptavidin on its own in the optical channel, and the carrier-streptavidin complex in both channels, FIG. 17. Detection of such low protein concentrations is not typical when sensing proteins natively without a carrier, due to fast translocation times and event rates often being significantly lower than predicted from the Smoluchowski rate equation, which often necessitate protein concentrations well in excess of 10s-100s nM [17]. Addition of the carrier, therefore, facilitates detection of a bound event via the synchronized electro-optical signal, FIG. 12 and FIG. 17. Similar to the previous example, binding of streptavidin to the carrier produces a substantial improvement in both the dwell time and total fluorescence intensities (e.g. 21.4±9.3 ms and 632±258 photons vs 3.1±2.0 ms and 92±47 photons respectively). Although previous studies [18-20] have demonstrated that the binding of protein to a long DNA carrier could be identified by reading out the sub-levels of the signal, fluorescence enables the direct quantification of a bound event and eliminates any possible false positives due to the influence on DNA conformational changes such as folds or knots [21-22]. For example, using our nanopore configuration, ^{˜}32% to ^{˜}36% of all events produced sub-peaks for both λ-DNA on its own and the biotinylated carrier. In contrast, 0% synchronisation was observed in the optical channel producing no false positives, FIG. 19.

Binding affinity can be determined from the synchronised fraction (the percentage of synchronised counts over all electrical counts) as a function of the streptavidin concentration, FIG. 12. As expected, the fraction of synchronised events increases with an increasing concentration of streptavidin. In this case, the carrier concentration was kept constant at 10 pM, and streptavidin was ramped from 0-100 pM. At 0 pM streptavidin, only events in the electrical channel were observed while at a 2× excess the synchronised fraction (85.7±2.2%) increased accordingly and reached a plateau representing the saturation of streptavidin bound to the biotinylated carrier. The Hill binding model, which typically describes the equilibrium state of reversible molecular binding, [23] could then be used to determine the apparent dissociation constant. Using this approach, a binding affinity of Kd ^{˜}7.6±1.2 pM was obtained. This value is roughly two orders of magnitude larger than that the wild-type streptavidin-biotin (10-14 M) [24] and comparable (10-8 to 10-11 M) [25] to cases where the associated may be affected by dye conjugation [26] or attachment of biotin moieties to a larger group can also restrict its free diffusing and thus reduce their affinity [27].

Label-Free Sensing Using Molecular Beacons Incorporated into the DNA Carriers

MBs are short oligonucleotides with a stem-loop structure, whose sequences could be designed as needed to specifically recognise a range of nucleic acids via hybridisation chemistry or proteins using aptamer sequences [11,28]. Instead of direct labelling of the targets, the fluorophore quencher pair was incorporated into the MB-Carrier. Fluorescence could then be restored upon binding to either a complementary strand, as shown in FIG. 20. The system could then be further extended to bind to other targets (for example proteins, FIG. 20) by incorporating aptamer sequences into the MBs [29].

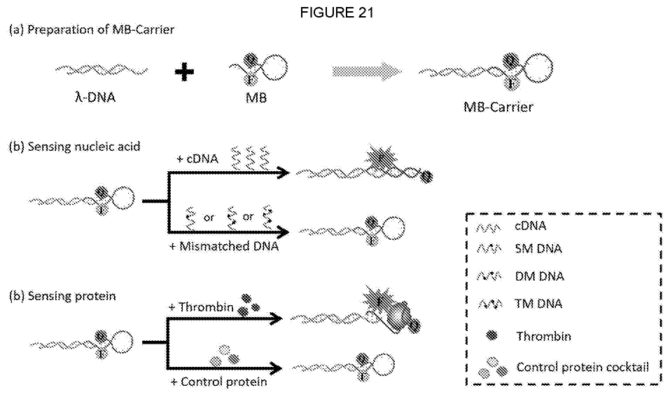

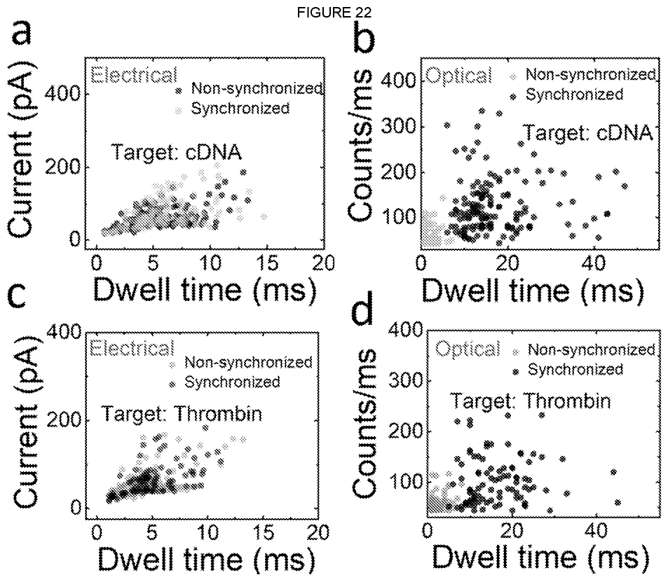

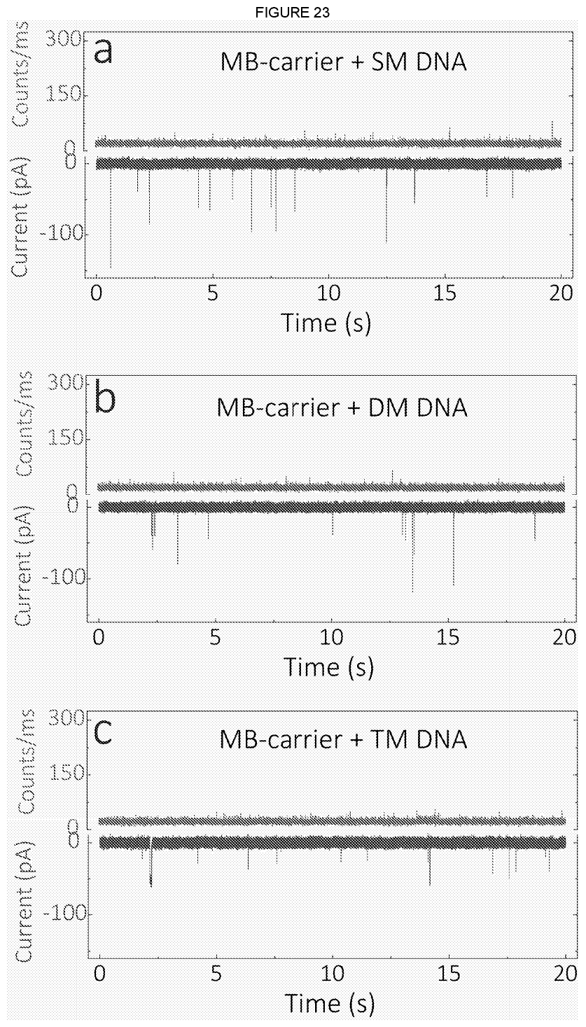

As an example, we incorporated a 15 mer thrombin-binding aptamer (TBA) [30] into the loop of the MB. TBA was selected due to its well-established structure, and high affinity towards thrombin (Kd ^{˜}2.68-200 nM) [30-32]. The design principle and sequence of this TBA-embedded MB and its incorporation into the λ-DNA to form a MB-carrier is described in detail in the methods section and FIG. 21. Translocation experiments of the MB-carriers, as well as their targets, were performed at an applied voltage of −300 mV. A series of three experiments were performed one with the MB-Carrier (10 pM) on its own (control), MB-Carrier (10 pM) including hybridisation of a complementary strand (15 mer: 5′-CCA ACC ACA CCA ACC-3′, 50 pM)(SEQ ID NO: 4) and finally MB-Carrier (10 pM) with the addition of thrombin (30 nM), FIG. 20. In the control, only the electrical signal was observed, while upon opening up of the MB whether it be from the complementary oligo or thrombin, the simultaneous electro-topical signal is observed. For DNA hybridisation, the MB loop undergoes structural transition when bound with target DNA to form a duplex state, resulting in the separation of the fluorophore from the quencher, FIG. 21. While for thrombin binding, the MB aptamer changes from its stem-loop shape to form a G-quadruplex structure upon binding, FIG. 21, extending the distance between the fluorophore and the quencher [28]. Apart from the synchronisation, the binding of MB-Carrier towards its targets has also led to significant enhancement in the dwell time and total photon counts, see FIG. 22, which further confirm and facilitate the identification of targets.

Interestingly, when zooming into the synchronised electro-optical events, two types of signals could be observed with either the electrical signal or the combined electrical-optical signal coming first, FIG. 20. This correlates to the two different orientations to the complex is transported through the pore. For example, in the “head to tail” configuration, the MB is transported through the pore first, while in the “tail to head” configuration, the carrier is transported first. To quantify the translocation orientation, we normalised the electrical dwell times with the start time being defined as 0 and the end time as 1. The fractional position of the optical signal relative to the electrical could then be plotted, FIG. 20, where two populations could be observed one at 0.003 and the other at 0.930 which correspond to the two possible orientations. Determining orientation is often difficult purely based on electrical data, however, a combined approach facilitates the extraction of this parameter. Much like the case for streptavidin, binding affinities could be determined including selectivity by characterising the percent of synchronised events, FIG. 20. For example, the selectivity of the MB-Carrier was compared to a corresponding complementary DNA strand (cDNA, 15 bases, 70.3±6.4%) along with similar length containing single (SM, 8.9±1.9%), double (DM, 3.9±1.7%), and triple (TM, 2.7±0.6%) base mismatches at a concentration of 50 pM, FIG. 20 and FIG. 23. Such selectivity gives rise to an 8-fold improvement in signal for the cDNA as compared to the SM which highlights the excellent capability of this approach for discrimination of single nucleotide polymorphism (SNP) without the need for amplification. By performing a titration and fitting using the Hill model, FIG. 20, the binding affinity was determined to be 3.7±0.2 pM for cDNA which is close to the value estimated from the Gibbs free energy (^{˜}0.9 pM), see note in SI. The detection limit was determined to be 0.1 pM, based on comparing the synchronised fraction to the blank control (0.78%±0.38%). It should be noted that this limit is lower than that more conventional single molecule counting methods (0.7 pM) [33] including two-colour coincident detection (0.5 pM) [34].

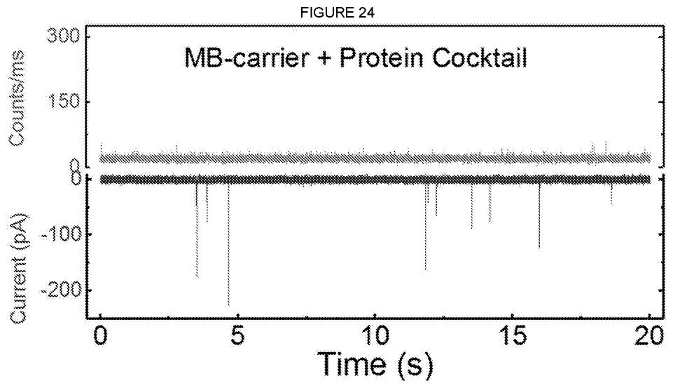

As a thrombin binding aptamer sequence was incorporated in the MB, a similar experiment could be performed with the addition of protein, FIG. 20. The thrombin selectivity at 30 nM was characterised by performing control experiments within a much more concentrated background (>300× excess for each target) containing a cocktail of proteins including lysozyme, trypsin, α-synuclein and insulin, FIG. 20 and FIG. 24. Importantly a 10-fold increase in the percent synchronised could be observed when comparing thrombin to the protein cocktail which highlights the excellent selectivity and possibility to discriminate between the target protein and other proteins. The binding affinity, FIG. 20, was determined to be 5.0±0.4 nM which is in perfect agreement with alternative approaches (4.87 to 10 nM) [28,29,35]. The detection limit for thrombin was determined to be 0.5 nM, which is also significantly lower than other reported methods based on single molecule methods [16,36].

Sensing of cDNA and Protein Targets in Human Serum and Urine

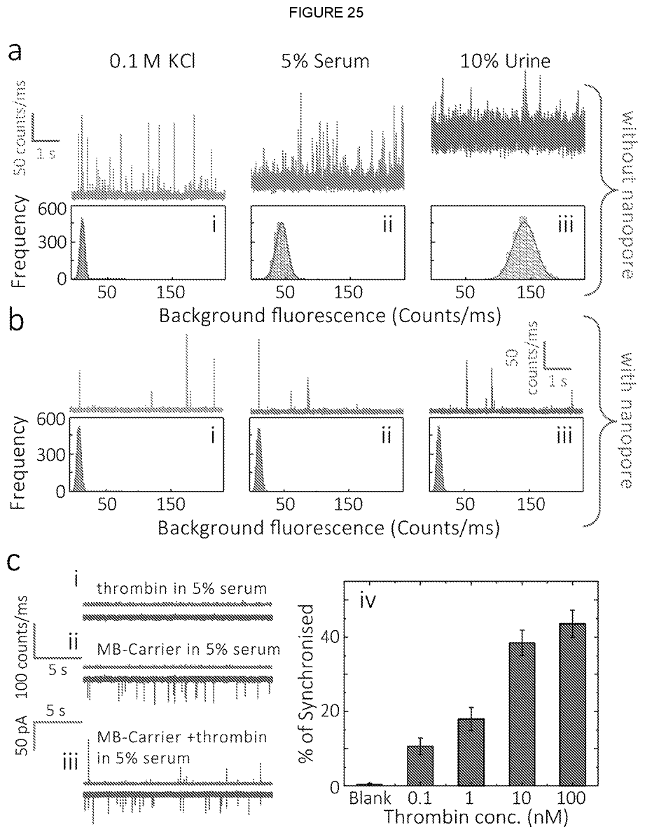

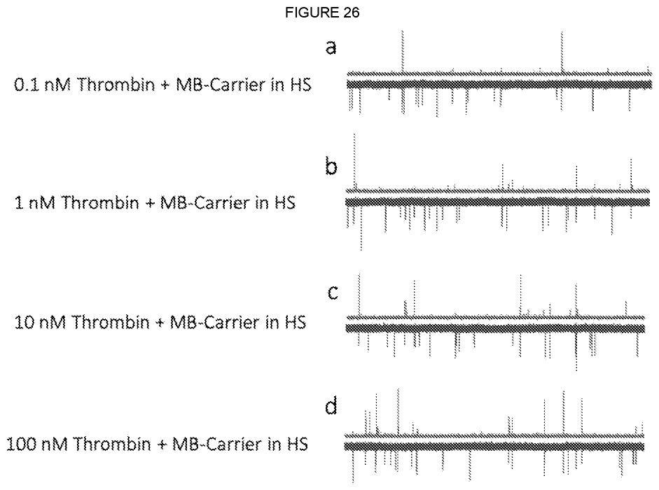

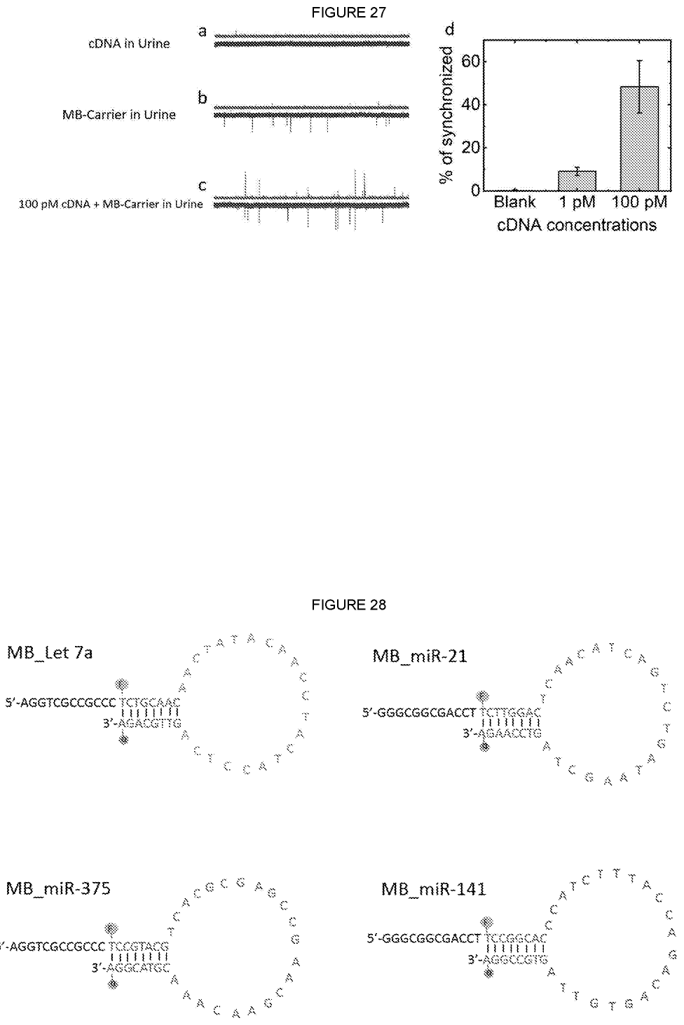

When using a conventional single molecule confocal fluorescence strategy (e.g. droplet on coverslip) FIG. 25 for detection of cDNA bound to the MB-Carrier, the background fluorescence clearly increases in both the serum and urine samples. However, when using a nanopore FIG. 25, the background fluorescence is almost identical to that of measurements taken in 0.1 M KCl. This is due to the sample being confined to within the nanopipette, the solution outside the nanopipette consists only of the KCl buffer. As can be seen this results in a substantial increase in signal to noise. An example of a binding assay in serum is shown in FIG. 25 and FIG. 26, the MB-Carrier concentration was 30 pM and thrombin was increased from 0.1-100 nM respectively and results are comparable to those obtained in 0.1 M KCl. An analogous study has also been performed in urine, FIG. 27.

It should be understood by the skilled person that the features of the various aspects and embodiments described herein can be combined with the features of the other various aspects and embodiments.

### REFERENCES (EXAMPLE 1)

[1]. Pitchford W H, et al. ACS Nano 9, 1740-1748 (2015).
[2] Singer A, Wanunu M, Morrison W, Kuhn H, Frank-Kamenetskii M, Meller A. Nano Lett. 10, 738-742 (2010).
[3]. Wanunu M, Dadosh T, Ray V, Jin J, McReynolds L, Drndić M. Nature Nanotechnology 5, 807 (2010).
[4] Liu S, Zhao Y, Parks J W, Deamer D W, Hawkins A R, Schmidt H. Nano Letters 14, 4816-4820 (2014).
[5]. Liu S, Wall T A, Ozcelik D, Parks J W, Hawkins A R, Schmidt H. Chemical Communications 51, 2084-2087 (2015).
[6]. N. A O, Tal G, Joshua S, Matyas J, Elmar W, Amit M. Advanced Materials 29, 1605442 (2017).
[7]. Gilboa T, et al. ACS Nano 10, 8861-8870 (2016).
[8]. Chansin G A T, Mulero R, Hong J, Kim M J, deMello A J, Edel J B. Nano Letters 7, 2901-2906 (2007).
[9]. Sawafta F, Clancy B, Carlsen A T, Huber M, Hall A R. Nanoscale 6, 6991-6996 (2014).
[10]. Larkin J, Foquet M, Turner S W, Korlach J, Wanunu M. Nano Letters 14, 6023-6029 (2014).
[11]. Tyagi S, Kramer F R. Nature Biotechnology 14, 303 (1996).
[12]. Guo B Y, Sheng Y Y, Zhou K, Liu Q S, Liu L, Wu H C. Angew Chem Int Edit 57, 3602-3606 (2018).
[13]. Freedman K J, Otto L M, Ivanov A P, Barik A, Oh S-H, Edel J B. Nature Communications 7, 10217 (2016).
[14]. Lin X, Ivanov A P, Edel J B. Chemical Science 8, 3905-3912 (2017).
[15]. Sze J Y Y, Kumar S, Ivanov A P, Oh S-H, Edel J B. Analyst 140, 4828-4834 (2015).
[16]. Sze J Y Y, Ivanov A P, Cass AEG, Edel J B. Nature Communications 8, 1552 (2017).
[17]. Plesa C, Kowalczyk S W, Zinsmeester R, Grosberg A Y, Rabin Y, Dekker C. Nano Letters 13, 658-663 (2013).
[18]. Bell N A W, Keyser U F. Journal of the American Chemical Society 137, 2035-2041 (2015).
[19]. Plesa C, Ruitenberg J W, Witteveen M J, Dekker C. Nano Letters 15, 3153-3158 (2015).
[20]. Yu J-S, et al. ACS Nano 9, 5289-5298 (2015).
[21]. Plesa C, et al. Nature Nanotechnology 11, 1093 (2016).
[22]. Steinbock U, Otto O, Chimerel C, Gornall J, Keyser U F. Nano Letters 10, 2493-2497 (2010).
[23]. Atkins P W, De Paula J. Atkins' Physical chemistry. Oxford University Press (2006).
[24]. Green N M. Avidin. In: Advances in Protein Chemistry (eds Anfinsen C B, Edsall J T, Richards F M). Academic Press (1975).
[25]. Huang S-C, Stump M D, Weiss R, Caldwell K D. Analytical Biochemistry 237, 115-122 (1996).
[26]. Jacobsen M T, Fairhead M, Fogelstrand P, Howarth M. Cell Chemical Biology 24, 1040-1047.e1044 (2017).
[27]. Fogen D, Wu S-C, Ng K K-S, Wong S-L. PLOS ONE 10, e0139137 (2015).
[28]. Li J J, Fang X, Tan W. Biochemical and Biophysical Research Communications 292, 31-40 (2002).
[29]. Hamaguchi N, Ellington A, Stanton M. Analytical Biochemistry 294, 126-131 (2001).
[30]. Bock L C, Griffin L C, Latham J A, Vermaas E H, Toole J J. Nature 355, 564 (1992).
[31]. Wu O, Tsiang M, Sadler J E. Journal of Biological Chemistry 267, 24408-24412 (1992).
[32]. Tasset D M, Kubik M F, Steiner W. Journal of Molecular Biology 272, 688-698 (1997).
[33]. Wang T-H, Peng Y, Zhang C, Wong P K, Ho C-M. Journal of the American Chemical Society 127, 5354-5359 (2005).
[34]. Neely L A, et al. Nature Methods 3, 41 (2005).
[35]. Heyduk E, Heyduk T. Analytical Chemistry 77, 1147-1156 (2005).
[36]. Rotem D, Jayasinghe L, Salichou M, Bayley H. Journal of the American Chemical Society 134, 2781-2787 (2012).

### Example 2: Simultaneous Single-Molecule Detection of Multiple microRNAs Using Nanopore and Fluorophore Detection; and Sensitivity Testing and One-Base Mismatch (Polymorphism) Differentiation/Detection

Methods

The nanopore set up and carrier construction utilised was the same as in Example 1 except where stated to be different.

Sequence of molecular beacons
MB_Let 7a: 5′ [symbol] T(Alexa 488) CTGCAAC AAC TAT ACA ACC TAC TAC CTC A
GTTGCAGA-Dabcyl-3′ (SEQ ID NO: 9); (Batch No. 181524)
MB_miR-21: 5′- [symbol] T(Alexa 488) CTTGGAC TCA ACA TCA GTC TGA TAA GCT A
GTCCAAGA-Dabcyl-3′ (SEQ ID NO: 10); (Batch No. 181525)
MB_miR-375: 5′- [symbol] T(Alexa 488) CCGTACG T CAC GCG AGC CGA ACG AAC AAA
CGTACGGA-Dabcyl-3′ (SEQ ID NO: 11); (Batch No. 182990)
MB_miR-141: 5′- [symbol] T(Alexa 488) CCGGCAC C CAT CTTTAC CAG ACA GTG TTA
GTGCCGGA-Dabcyl-3′ (SEQ. ID NO: 12). (Batch No. 182991)

The bases in italics and bold indicate the complementary part to the sticky overhang of λ-DNA.

The bases underlined form the stem of hairpin structure and the bases in bold (with no italics) form the loop that is complementary to corresponding microRNA targets. Schematics for these MBs are shown in FIG. 28.

TABLE 1
Sequences of DNA and microRNA oligos

| Oligos | Sequences | Batch No. | Specification |
|---|---|---|---|
| Let 7a_DNA | 5′-TGA GGT AGT AGG TTG TAT AGT | 285921257 | |
| | T-3′ (SEQ ID NO: 13) | | |
| miR-21_DNA | 5′-TAG CTT ATC AGA CTG ATG TTG | 287050822 | |
| | A-3′ (SEQ ID NO: 14) | | |
| Let 7a | 5′-UGA GGU AGU AGG UUG UAU | 290814212 | |
| | AGU U-3′ (SEQ ID NO: 15) | | |
| miR-21 | 5′-UAG CUU AUC AGA CUG AUG | 290814193 | |
| | UUG A-3′ (SEQ ID NO: 16) | | |
| miR-375 | 5′-UUU GUU GGU UCG GCU CGC | 295698629 | Prostate cancer |
| | GUG A-3′ (SEQ ID NO: 17) | | biomarker |
| miR-141 | 5′-UAA CAC UGU CUG GUA AAG | 295698735 | Prostate cancer |
| | AUG G-3′ (SEQ ID NO: 18) | | biomarker |
| Let 7f | 5′-UGA GGU AGU AGA UUG UGU | 296873668 | Single-mismatched with |
| | GGU U-3′(SEQID NO: 19) | | Let 7a |
| miR-200a | 5′-UAA CAC UGU CUG GUA ACG | 297280832 | Same family with miR- |
| | AUG U-3′ (SEQ ID NO: 20) | | 141 with double- |
| | | | mismatched |
| Scrambled miR- | 5′-AUG AGU GAG AGA UAC GCU | 296907839 | Scrambled sequence for |
| 141 | UCUA-3′ (SEQ ID NO: 21) | | miR-141 |

Digestion of Lambda-DNA and Preparation of MB-Engineered DNA Carriers

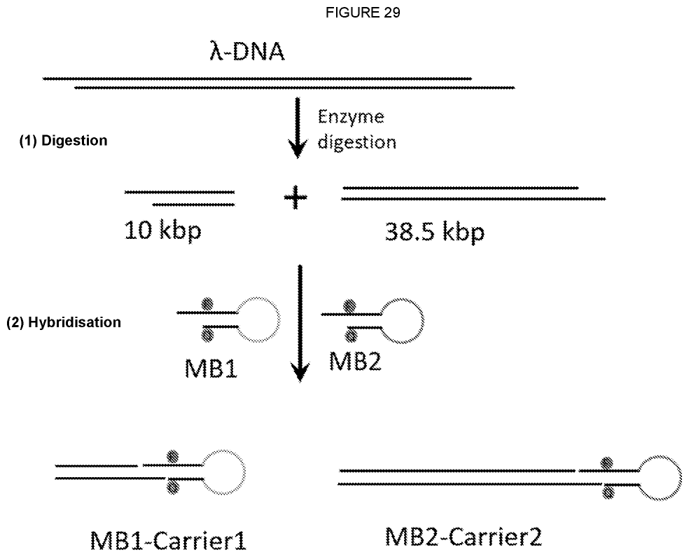

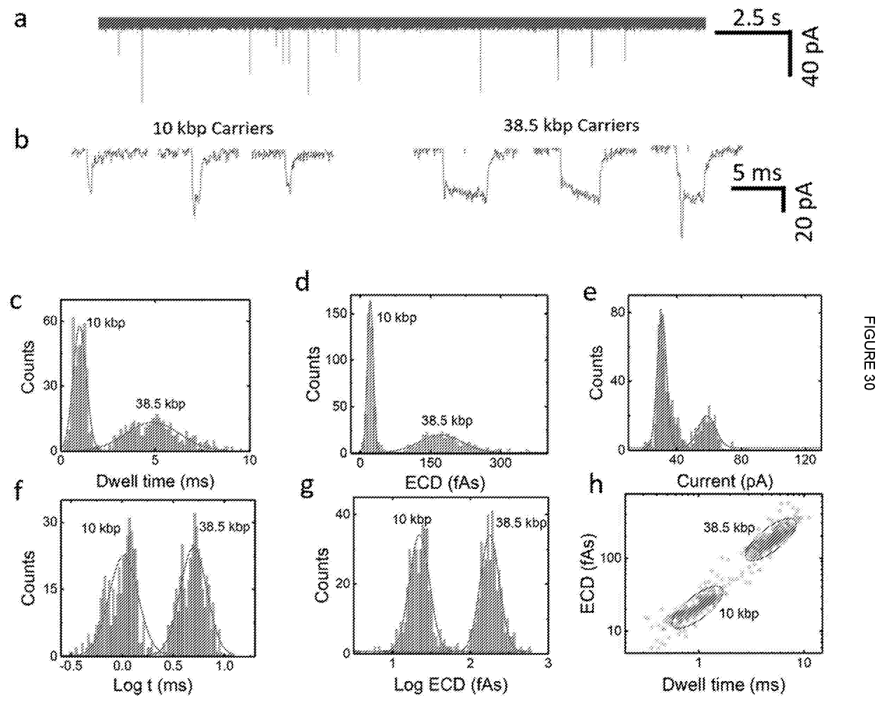

λ-DNA (48.5 kbp) was digested into two segments, 10 kbp and 38.5 kbp, using the digestion enzyme Apa I, according to the supplier's protocol. 12.5 μl of stock λ-DNA (15.8 nM), 5 μl of a 10× CutSmart buffer, 2.5 μl Apa I, and 30 μl of sterile water (Sigma-Aldrich) were first mixed to a final volume of 50 μl. This mixture was subsequently incubated at 25° C. for 30 minutes and then heated at 65° C. for 20 minutes to inactivate the enzyme. At this stage, the λ-DNA was digested into two fragments; one is 10 kbp, and the other is 38.5 kbp (FIG. 29).

To prepare the MB-engineered DNA carriers, 25 μl of 400 nM MB1 (to the 10 kbp fragment) and 25 μl of 400 nM MB2 (to the 38.5 kbp fragment) were added to the mixture obtained above with a fragment-to-MB ratio of 1:50. The hybridisation was run with a PCR annealing device (TECHNE, TC-3000). The mixture was heated to 75° C. for five minutes to denature the MB oligos and DNA fragments that digested from λ-DNA followed by the annealing procedure by decreasing the temperature to 15° C. at a rate of 1° C. per minute. The resulting products were held at 4° C. before purification (FIG. 29).

For the purification, excess unbound MB oligos, the remaining enzyme, and the BSA from the CutSmart buffer were removed using a commercially available filter kit, 100 kDa MWCO Amicon Ultra Filter (Millipore, UK). The solution was transferred into the filter column, and a solution of 100 mM KCl, 5 mM MgCl₂, and 10 mM TE (pH=8.0) was added to a total volume of 400 μl. The purification was then operated with six cycles of ultra-centrifuging at a speed of 3500×g at 4° C. for 30 minutes. The resulting product was recovered by turning the filter column upside down and centrifuging at 1000×g for two minutes. The final concentrations of carriers (both 10 kbp and 38.5 kbp) were determined by measuring the UV-Vis absorbance at 260 nm (as described above) and stored at −20° C. before use.

Serum Preparation

Whole blood from patients with different stages of prostate cancer was collected in 2014 following written patient consent using a standard venepuncture procedure and stored at the Tissue Biobank of Imperial College Healthcare NHS Trust (London, UK). For research purposes, the blood was then obtained from the biobank with ethical approval from patients who attend clinics at Imperial College Healthcare NHS Trust (London, UK) (REC Reference: 17/WA/0161). To prepare the serum, the blood in a red-topped vacutainer (silicon-coated with clot activator BD) was kept upright at room temperature for 30 to 60 minutes, allowing the blood to clot. Samples were then centrifuged at 1000-3000×g at room temperature for ten minutes, and the serum in the supernatant was isolated, aliquoted (1 ml per cryovial) and stored at −80° C. Prior to use, the serum was completely thawed at room temperature for approximately one hour.

microRNA/DNA Binding to MB-Carriers

Prior to translocation, MB-Carriers and microRNAs/DNA were diluted using a KCl buffer (100 mM KCl, 5 mM MgCl₂, 10 mM Tris-EDTA, pH=8.0) and mixed at different concentration ratios to incubate for two hours at a final carrier concentration of 10 amol μl⁻¹. For the experiments with patients' serum, MB-Carriers were diluted using a KCl buffer (100 mM KCl, 5 mM MgCl₂, 10 mM Tris-EDTA, pH=8.0) and subsequently mixed with human serum at a ratio of 20:1 at a final carrier concentration of 10 pM. The mixture was incubated for at least two hours prior to opto-electronic measurements.

Results

A designed MB sequence that specifically targets a miRNA was incorporated into the DNA carrier (MB-Carrier) to identify the presence of the target miRNA molecule. In the synchronised opto-electronic detection, the nanopore serves as a physical gate to deliver a carrier molecule into the aperture and monitors the transport by measuring the ionic current change, whereas the optical readout serves as the ‘Report’ signal to indicate the binding of the miRNA to the MB. For sensing multiple miRNAs, different lengths of the DNA carrier were assigned to encode the carriers for different miRNA targets. The length differentiation was characterised by individual electrical events from which the typical dwell time and peak area are proportional to the size of the DNA carriers. In the presence of target miRNAs, the MB on the carriers can be opened, and a corresponding fluorescence emission burst was observed, synchronously accompanied by the current spike. By quantifying the fraction of synchronised events over all the translocations of carriers, one can determine the concentrations of individual miRNAs.

Differentiation of Carriers

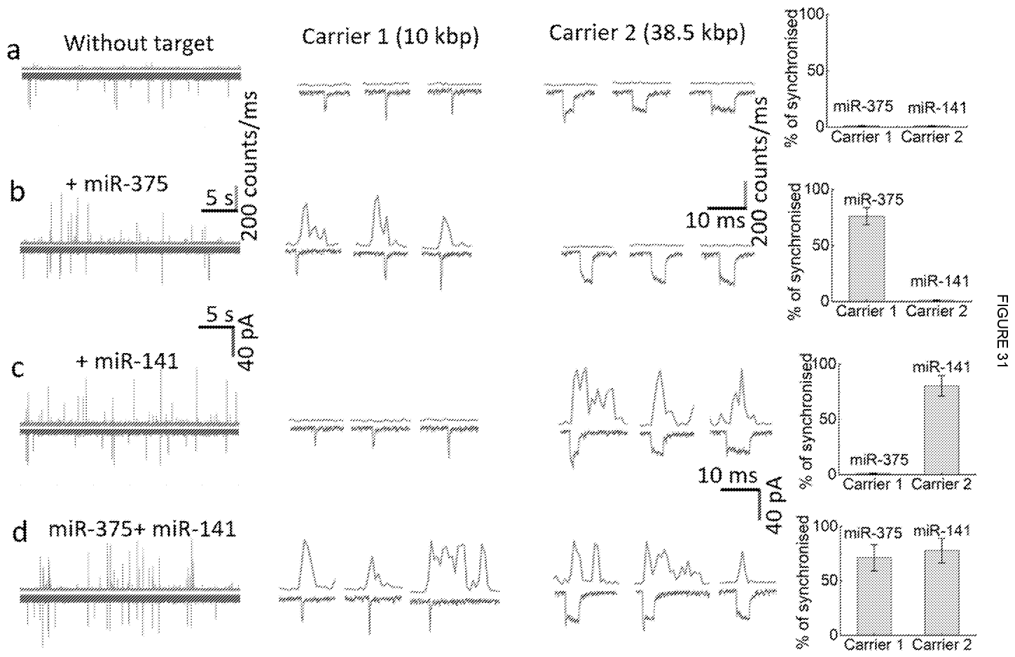

The ability of the nanopipette for electrically discriminating the two DNA carriers (10 kbp and 38.5 kbp) was examined. A translocation experiment was performed with both DNA fragments at concentrations of ^{˜}10 pM in a 100 mM KCl buffer (5 mM MgCl₂, 10 mM Tris-EDTA, pH=8.0) using a typical nanopipette (^{˜}20 nm). A continuous current trace at −300 mV shown in FIG. 30a reveals the typical translocation of DNA strands. A zoomed-in view of representative events, indicative of two populations, is shown in FIG. 30b. The events with smaller dwell time and peak area represent the transport of 10 kbp DNA carriers, while the wider events could be assigned to the transport of 38.5 kbp DNA. During the translocation, the DNA was assumed to be un-coiled to match the nanopore in the cross-section and then transported through the pore, displacing a portion of electrolytes, with a measurable change in the ionic current. Therefore, the dwell time for 38.5 kbp DNA carrier (4.9±2.8 ms) is observed to be longer than that for 10 kbp (1.0±0.7 ms) due to the longer residence time spent on threading through the pore (FIG. 30c). This is also revealed by the larger peak area (171±92 fAs) for 38.5 kbp than that for 10 kbp (21±12 fAs) (FIG. 30d). The peak area in the current change signal is often relative to the overall charges of the molecule in translocation, which is called an event charge deficit (ECD).¹ The distribution of current amplitudes shows the two populations peaked at 30.6±5.7 pA and 58.6±10.7 pA. The latter represents a portion of events that translocate through the nanopore with a folding state, as shown in FIG. 30e. The log-scale plots for the dwell time and peak areas (ECD) as well as the scatter plots of dwell time vs peak area show that a two-population of DNA translocations could be readily discriminated using the electrical recordings, as seen FIG. 30f-h. This discrimination for different lengths of DNA carriers enables them to be readily encoded with different probes and perform multiplexing for different targets simultaneously.

Simultaneous Single-Molecule Detection of Two microRNAs

Having demonstrated the electrical separation of different carrier probes, the viability of multiplexing was then verified using the developed opto-electronic nanopore and the MB-engineered carriers. In the first demonstration, two-target sensing was shown for the detection of two distinct miRNA sequences, miR-141 and miR-375. Sensing for another two miRNA molecules, Let 7a and miR-21, were also shown. Let 7a² and miR-21³ RNA are commonly observed RNA sequences that are involved in a series of tumour regulations and are frequently investigated as biomarkers for many cancers, such as lung, breast, ovarian and colorectal cancer.⁴⁻⁶ miR-141 and miR-375 are two miRNA sequences that are extensively observed through upregulation in the circulating blood in prostate cancer patients^{7,8,9} For the design of the MB embedded DNA carriers, the loop of the MB sequences was designed as complementary to the target miRNAs, as shown above, and was then hybridised to the sticky overhang of the digested DNA carriers. For example, the MB for miR-375 that was incorporated into the 10 kbp carrier was noted with MB-Carrier_{10kbp_miR-375}, and the MB for miR-141 that was attached to the 38.5 kbp carrier was noted with MB-Carrier_{38.5kbp_miR-141}.

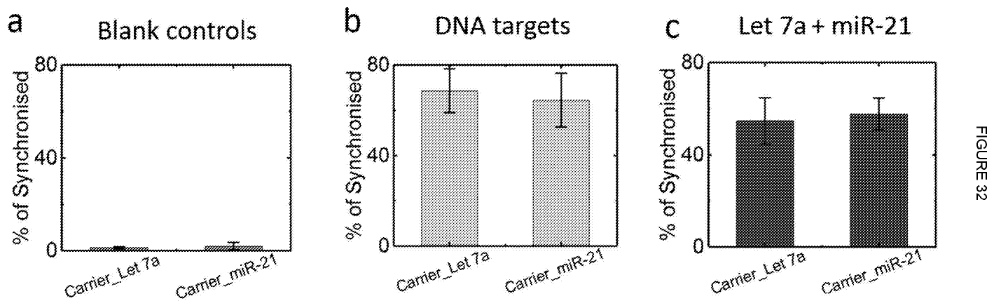

To test the sensing strategy, translocation experiments were performed using the MB-Carriers (concentrations are both 10 pM at the absence and presence of target miRNAs), and the resultant intensities-time traces were recorded (FIG. 31). For the MB-Carriers on their own, only the electrical signals were observed without any accompanying optical signal (FIG. 31a). From a more close-up view, two sets of electrical events, different in the dwell time and peak area, could be observed, corresponding to the transports of the smaller 10 kbp and the longer 38.5 kbp DNA carriers respectively (FIG. 31a). At this stage, because both the MB sequences are in the closed state, and the fluorescence is quenched, few synchronised optical events were observed, as indicated in the statistic of percentages synchronised (S). Then, the translocation of two MB-Carriers was performed with only the miR-375 present (equal molar). Translocations were observed in the electrical channel with some synchronised optical events (FIG. 31b). Based on the zoomed-in views of some typical events, most of the smaller electrical events were observed with synchronisation of optical signals, while longer electrical events were observed without corresponding photon bursts (FIG. 31b). The miR-375 bound to MB-Carrier_{10kbp_miR-375} to open the MB ‘stem-loop’ structure with fluorescence emission, while MB-Carrier_{38.5kbp_miR-141} remains closed. This is also revealed as the much higher percentage of synchronised events (S=76.1±7.6%) for the smaller carriers than those for the slower translocations (1.1±0.3%), FIG. 31b. In contrast, in the presence of sole miR-141, synchronised optical events were only observed with the longer translocation events, while the smaller carriers were recorded electrically. The corresponding S for the 38.5 kbp carrier is 80.5±9.2%, while the value for the 10 kbp carrier is negligible (FIG. 31c). At the presence of both miR-375 and miR-141, a higher portion of synchronised opto-electrical events from the intensities-time trances was observed, which indicates both carriers were bound to their target miRNAs, and the fluorescence from the MB was emitted. The S values for the miR-375 and miR-141 observed were reasonably high at 71.2±12.1% and 77.8±11.5%, respectively (FIG. 31d).

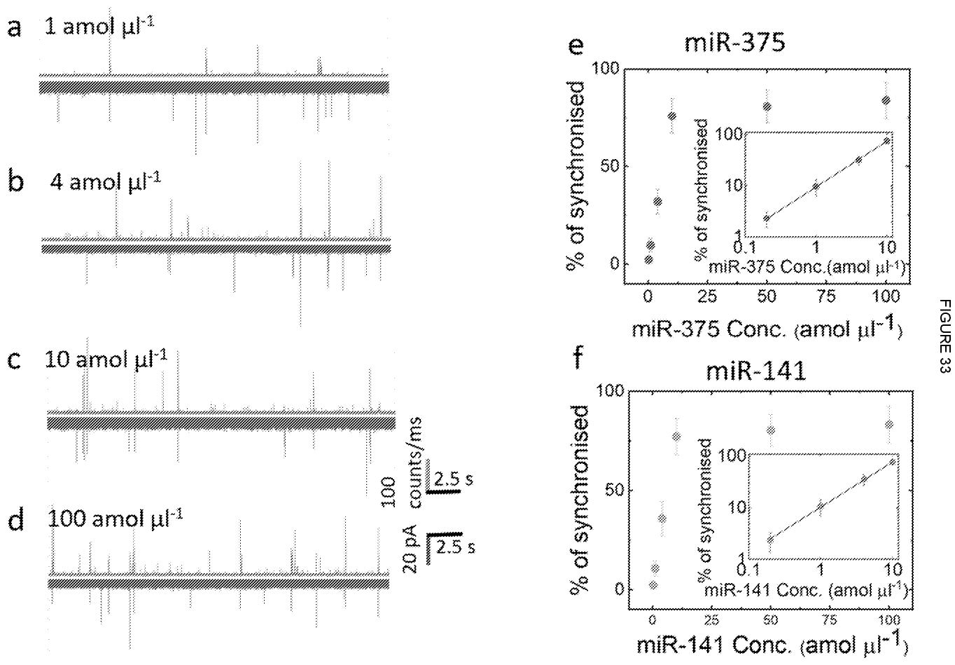

To confirm that the sensing strategy, the MB probes for miR-375 and miR-141 (MB_{_miR375}, MB_{_miR-141}) were replaced with two other MB probes for Let 7a and miR-21 (MB_{_Let 7a}, MB_{_miR-21}) Similar results were obtained for miRNA Let 7a and miR-21 as well as their DNA versions (FIG. 32). These, as well as the results obtained from miR-375 and miR-141, as described above, confirm the effectiveness of using the length-encoded DNA carriers for simultaneously probing multiple target miRNAs. Given the MB sequences could be designed as needed, one can sense other miRNAs sequences by adapting the complementary sequences into the loop of the hairpin structure of MB, allowing the technique to be extended at will to detect other important biomarkers.

Sensitivity and One-Base Mismatch Differentiation

Quantification and Sensitivity

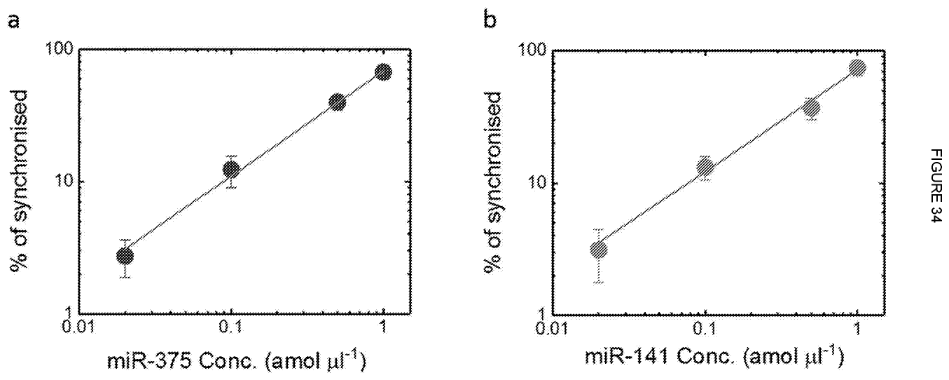

One of the advantages of synchronised detection is that the concentration of target molecules can be estimated by quantifying the percentage (5) of synchronised optical events over all electrical signals. The quantitative ability is demonstrated for two miRNA targets simultaneously. Calibration curves of S versus the increasing concentrations of synthetic miRNAs were first validated on the KCl buffer (100 mM KCl, pH=8.0). The initial concentration of Carrier_{10kbp_miR-375} and Carrier_{38.5kbp_miR-141} were kept constant at 10 pM throughout all experiments, while the concentration of both miR-375 and miR-141 increases from 0.2 pM to 100 pM. By increasing the miRNA concentration from 0.2 pM to 10 pM, the S first increased from 2.3±0.8% and 2.4±1.0% to 75.9±9.0% and 77.2±9.1%, respectively, and saturated at the miRNA concentration greater than 10 pM (FIG. 33). The log-scale S shows a linear relationship with the log-scale concentration of targets over a range of three orders (FIGS. 33e and f). The S at the lowest concentration (0.2 pM) was observed to be 2.3±0.8% and 2.4±1.0% for miR-375 and miR-141, respectively, over all translocation events of 833 and 669, which could be distinguished from the background noise (0.47±0.33% and 0.26±0.18% for shorter and longer translocations, respectively) of blank controls (total translocation events were 1275 and 1153).

Low Concentration miRNA Detection

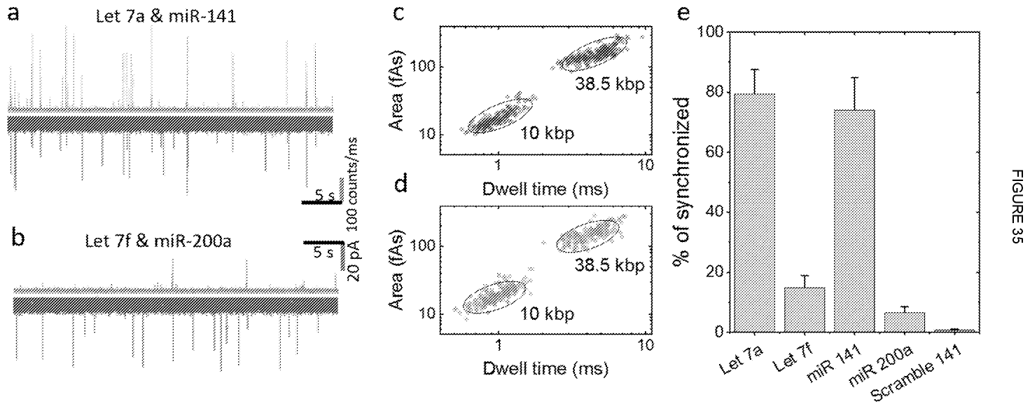

A salt gradient (40 mM/400 mM, cis/trans) was applied to a reduced concentration of carriers (1 pM) to improve the overall capture rate of translocation events. Calibration curves of S for both miR-375 and miR-141 were obtained with a linear increase from 0.02 pM to 1 pM (FIG. 34). The overall sample volume needed for the nanopore experiments could become as small as 1 μl, which indicates that as little as 0.02 amol (ca. 10,000 molecules) of miRNA could be accurately detected using this opto-electronic multiplexed platform. This ultra-sensitivity is particularly important for sensing miRNA in circulating biofluids where the copy number of miRNAs is often very

Single-Nucleotide Polymorphism Selectivity

It is challenging to discriminate close similarity between miRNAs (such as single-nucleotide polymorphisms) due to their inherent short length and high interference from the background, especially in biological samples. To demonstrate specificity, the prostate cancer-relevant miR-141, and its counterpart, miR-200a, were selected to test selectivity as they both belong to the miR-200 family and share 90.90% homology (20/22 bases). The other miRNA, miR-375, is found to have no close homologies according to the BLASTN, NCBI. Therefore, Let 7f was chosen as a control to the Let 7a due to a one-nucleotide mismatch and 95.45% homology.

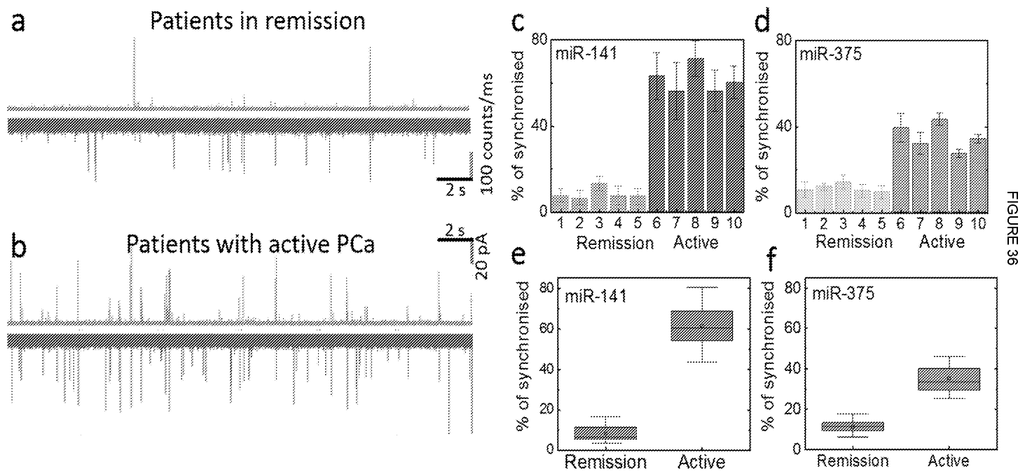

The fraction of synchronised events for the translocations of Carrier_{10kbp_Let 7a} and Carrier_{38.5kbp_miR-141} (10 pM) was compared at the presence of perfectly matched sequences Let 7a and miR-141, as well as the mismatched sequences, Let 7f and miR-200a. All the miRNAs were present at an equal molar concentration of 10 pM. As shown from the intensity-time trace (FIGS. 35a and b), the overall number of synchronised events for the perfectly matched targets (Let 7a and miR-141) were observed to be significantly greater than that for the mismatched targets (Let 7f and miR-200a). By analysing the electrical events, two populations were classified, which represent the two DNA carriers (FIGS. 35c and d). Further quantifying the fraction of synchronised events for each population, the fractions for the perfect targets, Let 7a and miR-141, were observed to be significantly higher, with 79.4±7.9% and 74.1±10.8%, respectively (FIG. 35e). As for the SNP, Let 7f, only 14.9±3.9% synchronisation was observed, revealing a factor of seven fewer than that of the Let 7a. The fraction decreases even lower to 6.6±1.8% when treated with a two-base mutant miR-200a. A scramble sequence to the miR-141 was also tested using this platform; however, only a negligible fraction (<1%) of synchronised events can be observed (FIG. 35e). The increasing mismatched bases add instability between the formed DNA-RNA duplexes and allow them to dissociate easily. These results above suggest the excellent specificity of this technique to differentiate similar sequences of miRNAs even for SNP discrimination.

Simultaneous Screening of miR-141 and miR-375 in Prostate Cancer Patients

miRNAs represent a new class of biomarkers that plays an essential role in post-transcriptional gene expression, and their aberrant expression is believed to offer correlations to early cancer stages. However, assessing cancer using single miRNA is difficult because the variation of expression in different disease stages might be very small and sometimes could overlap. Furthermore, one specific miRNA could act as a biomarker for multiple diseases rather than an indicator for a specific type of cancer. One possible way to improve the diagnostic effect is combining several miRNAs levels into a new class of indicator to determine the stage of a particular disease. Conventional technologies are challenging for profiling multiple miRNAs using a one-sample test and also need time-consuming, error-prone reverse amplification or pre-treatment.

Screening stages of prostate cancer (PCa) was examined by simultaneously quantifying multiple miRNAs levels directly from clinical samples (i.e. serum). MiR-141 and miR-375 are two typical miRNAs that have been reported to be upregulated in the tumour or circulation of Pca patients.^{7,8,9} As an example, these two miRNAs were selected as the targets to demonstrate the diagnostic value of this strategy.

Serum was collected from two groups of patients; people who have active cancer and people who are in remission. Prior to the test, prepared DNA carriers (Carrier_{10kbp_miR-375} and Carrier_{38.5kbp_miR-141}) were diluted using a KCl buffer (100 mM KCl, 5 mM MgCl₂, 10 mM Tris-EDTA, pH=8.0) and mixed with the untreated human serum at a ratio of 10:1 (with a final carrier concentration of 10 pM), followed by incubation at room temperature for approximately two hours. Then, the translocation experiments were performed in the presence of serum from patients in remission and active stages by loading ^{˜}1 μl of the above incubation inside the nanopipette. Intensities-time traces were recorded as shown in FIG. 36a-b. At first glance, a much higher frequency of synchronised opto-electrical events was observed for patients with active cancer than for patients in remission. To quantify the expression levels of both miR-141 and miR-375 for cancer patients and the remission group, the translocations were analysed in detail to assign different carrier signals and calculated the corresponding fraction of synchronised events (5). As expected, the average fractions of both miR-141 and miR-375 for patients in active Pca (n=5) are observed to be greater than those for people who are in remission (n=5) (FIGS. 36c and d). The mean levels of miR-141 and miR-375 expression for PCa patients were observed to increase by a factor of 6.56 and 3.1, respectively, compared to the patients who are in remission (FIGS. 36e and f).

### REFERENCES (EXAMPLE 2)

(1) Cadinu, P.; Paulose Nadappuram, B.; Lee, D. J.; Sze, J. Y. Y.; Campolo, G.; Zhang, Y.; Shevchuk, A.; Ladame, S.; Albrecht, T.; Korchev, Y.; et al. Single Molecule Trapping and Sensing Using Dual Nanopores Separated by a Zeptoliter Nanobridge. Nano Lett. 2017, 17 (10), 6376-6384.
(2) Ambros, V. MicroRNAs. Cell 2001, 107 (7), 823-826.
(3) Krichevsky, A. M.; Gabriely, G. MiR-21: A Small Multi-Faceted RNA. J. Cell. Mol. Med. 2008, 13 (1), 39-53.
(4) Toiyama, Y.; Takahashi, M.; Hur, K.; Nagasaka, T.; Tanaka, K.; Inoue, Y.; Kusunoki, M.; Boland, C. R.; Goel, A. Serum MiR-21 as a Diagnostic and Prognostic Biomarker in Colorectal Cancer. JNCI J. Natl. Cancer Inst. 2013, 105 (12), 849-859.
(5) Taylor, D. D.; Gercel-Taylor, C. MicroRNA Signatures of Tumor-Derived Exosomes as Diagnostic Biomarkers of Ovarian Cancer. Gynecol. Oncol. 2008, 110 (1), 13-21.
(6) Khalighfard, S.; Alizadeh, A. M.; Irani, S.; Omranipour, R. Plasma MiR-21, MiR-155, MiR-10b, and Let-7a as the Potential Biomarkers for the Monitoring of Breast Cancer Patients. Sci. Rep. 2018, 8 (1), 17981.
(7) Mitchell, P. S.; Parkin, R. K.; Kroh, E. M.; Fritz, B. R.; Wyman, S. K.; Pogosova-Agadjanyan, E. L.; Peterson, A.; Noteboom, J.; O'Briant, K. C.; Allen, A.; et al. Circulating MicroRNAs as Stable Blood-Based Markers for Cancer Detection. Proc. Natl. Acad. Sci. 2008, 105 (30), 10513-10518.
(8) Brase, J. C.; Johannes, M.; Schlomm, T.; Fälth, M.; Haese, A.; Steuber, T.; Beissbarth, T.; Kuner, R.; Sültmann, H. Circulating MiRNAs Are Correlated with Tumor Progression in Prostate Cancer. Int. J. Cancer 2011, 128 (3), 608-616.
(9) Metcalf, G. A. D.; Shibakawa, A.; Patel, H.; Sita-Lumsden, A.; Zivi, A.; Rama, N.; Bevan, C. L.; Ladame, S. Amplification-Free Detection of Circulating MicroRNA Biomarkers from Body Fluids Based on Fluorogenic Oligonucleotide-Templated Reaction between Engineered Peptide Nucleic Acid Probes: Application to Prostate Cancer Diagnosis. Anal. Chem. 2016, 88 (16), 8091-8098.
(10) Wang, H.; Peng, R.; Wang, J.; Qin, Z.; Xue, L. Circulating MicroRNAs as Potential Cancer Biomarkers: The Advantage and Disadvantage. Clin. Epigenetics 2018, 10 (1), 59.
(11) Dong, H.; Lei, J.; Ding, L.; Wen, Y.; Ju, H.; Zhang, X. MicroRNA: Function, Detection, and Bioanalysis. Chem. Rev. 2013, 113 (8), 6207-6233.

1. A method of detecting one or more analytes in a sample, the method comprising:
a. providing at least one carrier nucleic acid molecule comprising at least one single-stranded region;
b. providing at least one detection element comprising:
i. at least one fluorophore;
ii. at least one fluorescence quencher that quenches spectroscopic detection of the fluorophore;
iii. at least one analyte-binding moiety; and
iv. at least one nucleic acid moiety that binds to a single stranded region on the carrier nucleic acid molecule;
v. wherein the detection element is configured such that in the absence of the analyte the fluorophore is quenched by the fluorescence quencher and upon analyte binding to the analyte-binding moiety fluorescence is restored;
c. contacting the carrier nucleic acid molecule and detection element with the sample to form a carrier nucleic acid molecule/detection element/analyte complex;
d. providing a nanopore through which the carrier nucleic acid/detection element/analyte complex may be translocated;
e. translocating the carrier nucleic acid/detection element/analyte complex through the nanopore via voltage-driven translocation and monitoring time-dependent current response;
f. irradiating the nanopore with radiation that excites the fluorophore and monitoring radiation emissions of the fluorophore over time; and
g. comparing the signals from time-dependent current response and emission over time;
wherein a simultaneous signal in both time-dependent current response and emission over time indicates the binding of the analyte to the detection element.

2. The method of claim 1, wherein the detection element comprises a molecular beacon (MB).

3. The method of claim 1, wherein the number of detection elements corresponds to the number of the single stranded regions of the at least one carrier nucleic acid molecule.

4. The method of claim 1, wherein the analyte-binding moiety is an aptamer.

5. The method of claim 1, wherein the nanopore is at the tip of a nanopipette.

6. The method of claim 5, wherein the nanopipette is a quartz nanopipette.

7. The method of claim 1, wherein the carrier nucleic acid comprises lambda DNA.

8. The method of claim 1, in which:
i. the carrier nucleic acid has at least two single stranded regions; and
ii. a number of detection elements corresponding to the number of single stranded regions is provided;
wherein the analyte-binding moieties in each detection element may bind to the same or to different analytes and wherein each detection element has a different fluorophore.

9. The method of claim 8, wherein the analyte-binding moieties in each detection element bind to different analytes.

10. The method of claim 1, wherein the one or more analytes comprise DNA or RNA.

11. The method of claim 1, wherein the one or more analytes comprises a microRNA (miRNA).

12. The method of claim 11, wherein the miRNA is one or more of miR-141, miR-375, Let 7a and/or miR-21.

13. The method of claim 1, wherein the one or more analytes are cancer biomarkers.

14. The method of claim 13, wherein the cancer is selected from one or more of lung, breast, ovarian, colorectal and/or prostate cancer.

15. The method of claim 1, wherein the sample is human serum.

16. The method of claim 1, wherein the analyte-binding moiety comprises a nucleic acid, the sample is a control sample and the one or more analytes comprise a control nucleic acid comprising a sequence complimentary to the nucleic acid sequence of the at least one analyte-binding moiety; and wherein the method further comprises:
repeating steps a. to g. with a second sample wherein the one or more analytes comprise a target nucleic acid;
calculating a percentage of the occurrences of the simultaneous signal in both time-dependent current response and emission over time over all electrical signals obtained for the at least one carrier nucleic acid/detection element/control nucleic acid complex (S);
calculating a percentage of the occurrences of the simultaneous signal in both time-dependent current response and emission over time over all electrical signals obtained for the at least one carrier nucleic acid molecule/detection element/target nucleic acid complex (S′);
wherein a value of S′ lower than the value of S indicates the presence of one or more mutations and/or nucleotide polymorphisms in the target nucleic acid.

17. A method of quantifying a concentration of an analyte in a sample, the method comprising:
a. providing at least one carrier nucleic acid molecule comprising at least one single-stranded region;
b. providing at least one detection element comprising:
i. at least one fluorophore;
ii. at least one fluorescence quencher that quenches spectroscopic detection of the fluorophore;
iii. at least one analyte-binding moiety; and
iv. at least one nucleic acid moiety that binds to a single stranded region on the carrier nucleic acid molecule;
v. wherein the detection element is configured such that in the absence of the analyte the fluorophore is quenched by the fluorescence quencher and upon analyte binding to the analyte-binding moiety fluorescence is restored;
c. contacting the carrier nucleic acid molecule and detection element with a sample comprising an analyte to form a carrier nucleic acid molecule/detection element/analyte complex;
d. providing a nanopore through which the carrier nucleic acid/detection element/analyte complex may be translocated;
e. translocating the carrier nucleic acid/detection element/analyte complex through the nanopore via voltage-driven translocation and monitoring time-dependent current response;
f. irradiating the nanopore with radiation that excites the fluorophore and monitoring radiation emissions of the fluorophore over time; wherein a simultaneous signal in both time-dependent current response and emission over time indicates the binding;
g. comparing the signals from time-dependent current response and emission over time;
wherein a simultaneous signal in both time-dependent current response and emission over time indicates the binding of the analyte to the detection element;
h. calculating a percentage of the occurrences of the simultaneous signal in both time-dependent current response and emission over time over all electrical signals (S); and
i. comparing S to one or more reference values of S to determine the concentration of analyte.

18. The method of claim 17, wherein the one or more reference values of S are obtained by:
j. carrying out steps a. to h. wherein the sample is a control sample comprising a known concentration of the analyte; and
repeating step j. at least two times, wherein the known concentration of analyte is increased or decreased.

19. The method of claim 1, wherein the method comprises providing at least two carrier nucleic acid molecules, and wherein each carrier nucleic acid molecule has a different molecular weight and/or length.

20. The method of claim 19, wherein the method comprises providing at least two detection elements;
wherein at least one nucleic acid moiety of each detection element binds to a respective single stranded region of each of the at least two carrier nucleic acid molecules; and
wherein the at least one analyte-binding moieties in each detection element bind to different analytes.

21. An apparatus device for detection of an analyte characterised in that it is adapted to use the method of claim 1.

22. The apparatus of claim 21, comprising:
at least one volume for receiving a sample;
at least one nanopore, adapted to be in contact with the at least one volume for receiving a sample;
at least one source of potential difference, adapted to apply a potential difference across the at least one nanopore;
means for monitoring the time-dependent current response from the nanopore;
at least one source of electromagnetic radiation adapted to illuminate the at least one nanopore;
at least one detection means adapted to detect fluorescence radiation signals arising from the at least one nanopore; and
means for the comparison of signals from the means for monitoring the time-dependent current response from the nanopore and the signals form the at least one detection means, adapted to identify simultaneous events.

23. The method of claim 17, wherein the method comprises providing at least two carrier nucleic acid molecules, and wherein each carrier nucleic acid molecule has a different molecular weight and/or length.

24. The method of claim 23, wherein the method comprises providing at least two detection elements;
wherein at least one nucleic acid moiety of each detection element binds to a respective single stranded region of each of the at least two carrier nucleic acid molecules; and
wherein the at least one analyte-binding moieties in each detection element bind to different analytes.
